(19) United States
(12) Patent Application Publication
(10) Pub. No.: US 2019/0202814 A1
(43) Pub. Date: Jul. 4, 2019

(54) PROCESSES FOR PREPARATION OF EMPAGLIFLOZIN

(71) Applicant: CADILA HEALTHCARE LIMITED, AHMEDABAD (IN)

(72) Inventors: Sanjay Jagdish DESAI, Ahmedabad (IN); Jayprakash Ajitsingh PARIHAR, Ahmedabad (IN); Mahesh Laljibhai RUPAPARA, Ahmedabad (IN); Pranav Jitendra GANGWAR, Ahmedabad (IN); Hardik Bhikhubhai GHODASARA, Ahmedabad (IN)

(73) Assignee: CADILA HEALTHCARE LIMITED, AHMEDABAD (IN)

(21) Appl. No.: 16/295,199

(22) Filed: Mar. 7, 2019

Related U.S. Application Data
(63) Continuation of application No. 15/249,869, filed on Aug. 29, 2016.

(30) Foreign Application Priority Data
Nov. 9, 2015 (IN) 4286/MUM/2015
Dec. 22, 2015 (IN) 4815/MUM/2015

(51) Int. Cl. C07D 405/12; C07C 37/055; C07C 43/225; C07D 309/10; C07D 207/16; C07C 49/10

(52) U.S. Cl. CPC C07D 405/12; C07C 37/055; C07C 43/225; C07B 2200/13; C07D 207/16; C07C 49/10; C07D 309/10

(57) ABSTRACT

The present invention relates to processes for the preparation of empagliflozin. In particular, the present invention relates to the preparation of empagliflozin and intermediates thereof. The present invention also relates to co-crystal of empagliflozin and amino acid and amorphous form of empagliflozin.

### CROSS REFERENCE APPLICATIONS

This application is a continuation of U.S. patent application Ser. No. 15/249,869 filed Aug. 29, 2016, which claims the benefit of Indian Applications No.: 4286/MUM/2015 filed Nov. 9, 2015 and 4815/MUM/201 S filed Dec. 22, 2015 the content of which are incorporated herein by reference.

### FIELD OF THE INVENTION

The present invention relates to processes for the preparation of empagliflozin. In particular, the present invention relates to the preparation of empagliflozin and intermediates thereof. The present invention also relates to co-crystal of empagliflozin and amino acid and amorphous form of empaglitlozin.

### BACKGROUND OF THE INVENTION

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

Jardiance® (Empagliflozin) is a novel, orally administered, potent, and selective SGLT2 inhibitor indicated as an adjunct to diet and exercise to improve glycemic control in adults with type-2 diabetes mellitus. The chemical name of empagliflozin is (1S)-1, 5-anhydro-1-{4-chloro-3-(4-[(3S)-tetrahydrofuran-3-yloxy] benzyl} phenyl)-D-glucitol. It is also known as 1-chloro-4-(β-D-glucopyranos-1-yl)-2-[4-((S)-tetrahydrofuran-3-yloxy)-benzyl]-benzene and has the following structure:

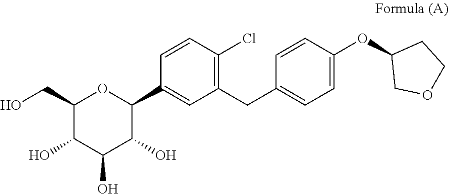 [SMILES: OC[C@H]1O[C@@H](c2ccc(Cl)c(Cc3ccc(O[C@H]4CCOC4)cc3)c2)[C@H](O)[C@@H](O)[C@@H]1O]

Empagliflozin is a white to yellowish non-hygroscopic crystalline solid, very slightly soluble in water, acetonitrile and ethanol, sparingly soluble in methanol, and practically insoluble in toluene.

U.S. Pat. No. 7,579,449 discloses empagliflozin, stereoisomers of empagliflozin, mixtures and salts thereof and pharmaceutical composition containing empagliflozin.

U.S. Pat. No. 7,713,938 discloses a crystalline form of empagliflozin and method for making the crystalline form.

U.S. Pat. No. 8,802,842 discloses method for preparing a crystalline form of compound 1-chloro-4-(β-D-glucopyranos-1-yl)-2-[4-((S)-tetrahydrofuran-3-yl oxy)-benzyl]-benzene.

International (PCT) Publication No. WO 2006/120208 discloses various methods of synthesis of SGLT2 inhibitors and also 1-chloro-4-(β-D-glucopyranos-1-yl)-2-[4-((S)-tetrahydrofuran-3-yloxy)-benzyl]-benzene. The publication also discloses novel intermediates and processes for their preparation.

International (PCT) Publication No. WO 2015/101916A1 discloses process for the preparation of empagliflozin and also discloses novel intermediates in the preparation of empagliflozin.

International (PCT) Publication No. WO 2016/051368 A1 discloses an amorphous empagliflozin complex with a cyclodextrin and process for the preparation thereof.

Chinese Publication CN 104788438 discloses empagliflozin B crystal form and process for preparation thereof.

The processes disclosed in the literature involve several chemical steps and provide the product in a very low overall yield and result into expensive processes for the preparation of the intermediate and the final empagliflozin API. The present invention provides multiple improvements over the prior art methods and also provides robust method with which empagliflozin may be obtained in high purity, with a low content of certain impurities and which allows the manufacturing in a commercial scale with a low expenditure and a high yields.

The processes disclosed in prior art references do not disclose co-crystals and processes for their preparation. Crystalline forms are often disadvantageous due to poor solubility, hygroscopicity, dissolution rate, and other associated performance characteristics. Co-crystallization can be used to purify a drug substance during manufacturing to reduce impurities and to obtain better quality of the API. The present invention also provides method for the preparation of co-crystal of empagliflozin with amino acid which allows the manufacturing of the drug substance with high purity and high yields on large scale production. The present invention also provides preparation of amorphous form of empagliflozin from the co-crystal.

### SUMMARY OF THE INVENTION

In one general aspect, there is provided a process for the preparation of empagliflozin, the process comprising:
(a) reacting 5-bromo-2-chlorobenzoic acid of Formula (X) with ethoxy benzene to obtain 5-bromo-2-chloro-4′-ethoxybenzophenone of Formula (IX);

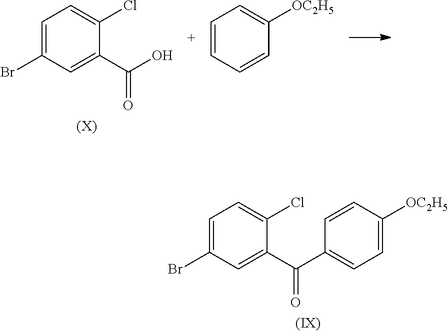

(b) reacting 5-bromo-2-chloro-4′-ethoxybenzophenone of Formula (IX) with reducing reagent to obtain 5-bromo-2-chloro-4′-ethoxydiphenylmethane of Formula (VIII);

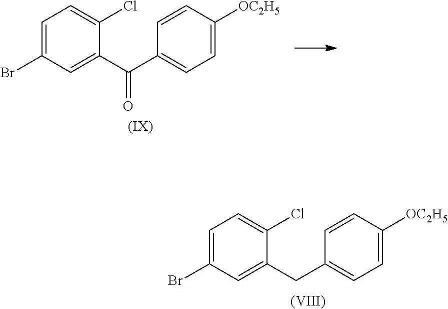

(c) reacting 5-bromo-2-chloro-4′-ethoxydiphenylmethane of Formula (VIII) with Lewis acid to obtain 5-bromo-2-chloro-4′-hydroxydiphenylmethane of Formula (VII);

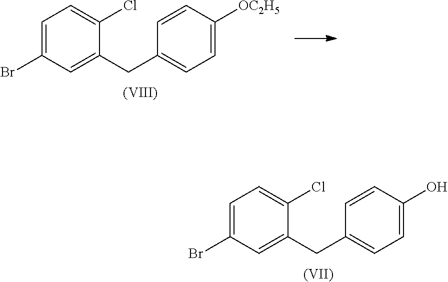

(d) reacting 5-bromo-2-chloro-4′-hydroxydiphenylmethane of Formula (VII) with hydroxy protecting reagent to obtain a compound of Formula (VI);

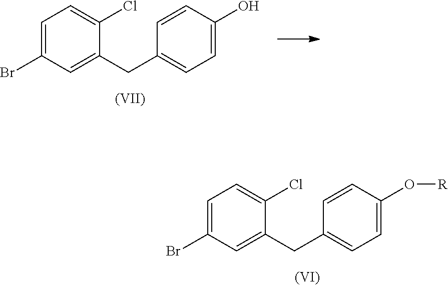

where R is a protecting group selected from trityl, allyl or tetrahydropyran,
(e) reacting the compound of Formula (VI) with a compound of Formula (P-G) to obtain a compound of Formula (V);

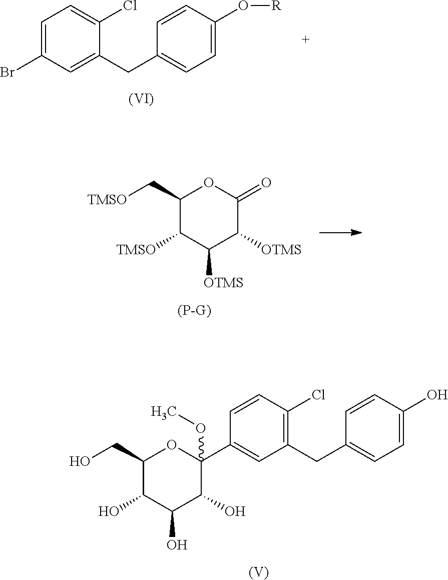

(f) reacting the compound of Formula (V) with a reducing reagent to obtain a compound of Formula (IV);

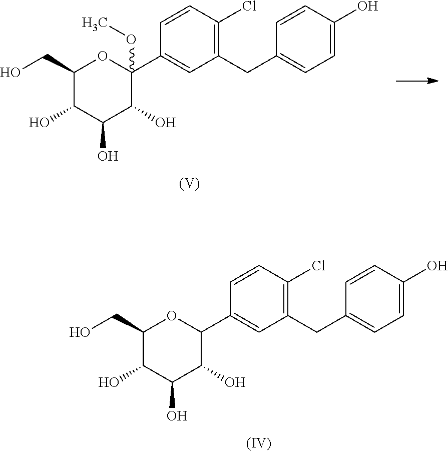 [SMILES: COC1(c2ccc(Cl)c(Cc3ccc(O)cc3)c2)O[C@H](CO)[C@@H](O)[C@H](O)[C@H]1O ; "(V)" ; OC[C@H]1OC(c2ccc(Cl)c(Cc3ccc(O)cc3)c2)[C@H](O)[C@@H](O)[C@@H]1O ; "(IV)"]

(g) reacting the compound of Formula (IV) with a hydroxy protecting reagent to obtain a compound of Formula (III);

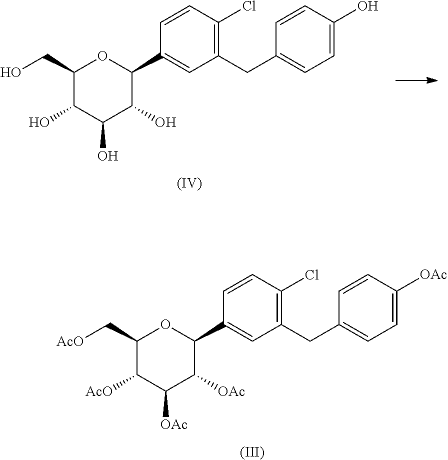 [SMILES: OC[C@H]1O[C@@H](c2ccc(Cl)c(Cc3ccc(O)cc3)c2)[C@H](O)[C@@H](O)[C@@H]1O ; "(IV)" ; CC(=O)OC[C@H]1O[C@@H](c2ccc(Cl)c(Cc3ccc(OC(C)=O)cc3)c2)[C@H](OC(C)=O)[C@@H](OC(C)=O)[C@@H]1OC(C)=O ; "(III)"]

(h) converting the compound of Formula (III) into a compound of Formula (II); and

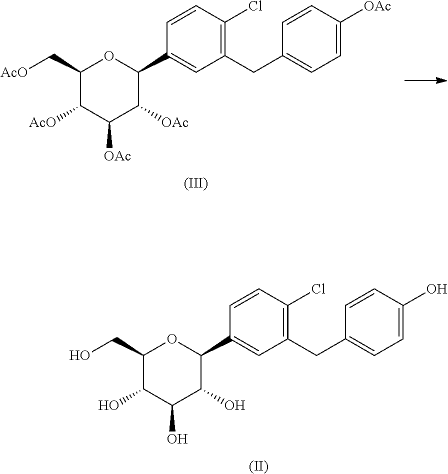 [SMILES: CC(=O)OC[C@H]1O[C@@H](c2ccc(Cl)c(Cc3ccc(OC(C)=O)cc3)c2)[C@H](OC(C)=O)[C@@H](OC(C)=O)[C@@H]1OC(C)=O ; "(III)" ; OC[C@H]1O[C@@H](c2ccc(Cl)c(Cc3ccc(O)cc3)c2)[C@H](O)[C@@H](O)[C@@H]1O ; "(II)"]

(i) converting the compound of Formula (HI) into empagliflozin.

In another general aspect, there is provided a process for the preparation of empagliflozin, the process comprising:
(a) reacting 5-bromo-2-chloro-4′-ethoxydiphenylmethane of Formula (VIII) with to Lewis acid to obtain 5-bromo-2-chloro-4′-hydroxydiphenylmethane of Formula (VII);

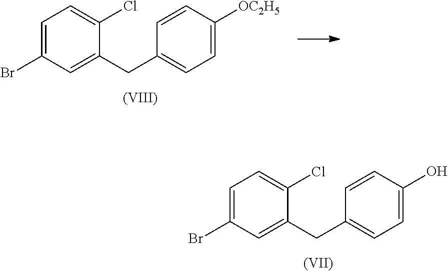

(b) reacting 5-bromo-2-chloro-4′-hydroxydiphenylmethane of Formula (VII) with a hydroxy protecting reagent to obtain a compound of Formula (VI);

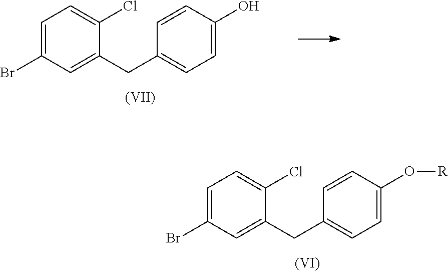

(c) reacting the compound of Formula (VI) with a compound of Formula (P-G) to obtain a compound of Formula (V);

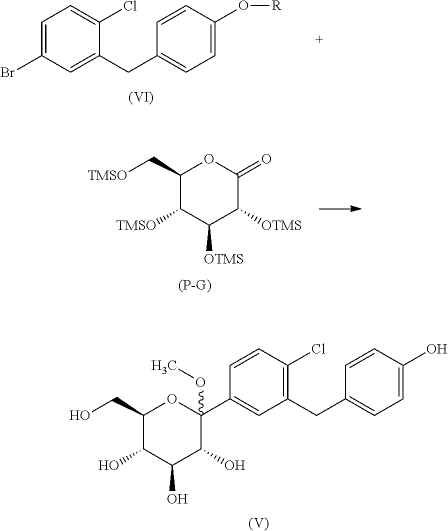

(d) converting the compound of Formula (V) into a compound of Formula (II-SH);

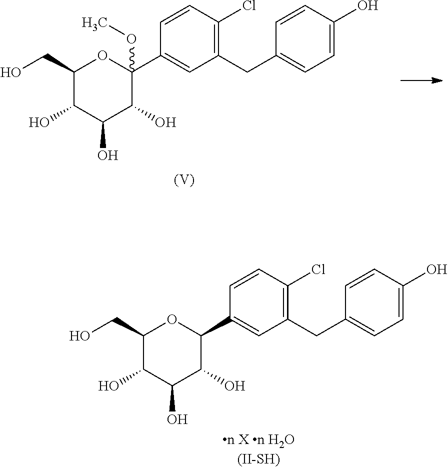

wherein n is 0.5 to 2, X is C₃ to C₆ ketone,
(e) convening the compound of the Formula (II-SH) into a compound of Formula (C); and

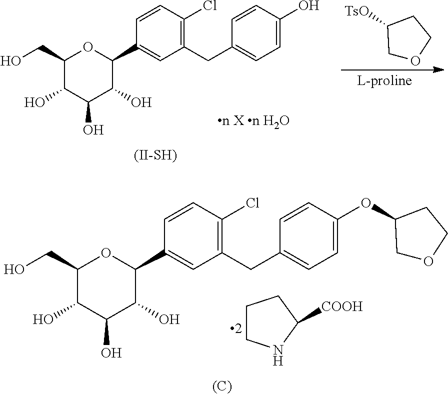

(f) converting the compound of Formula (C) into empagliflozin.

In another general aspect, there is provided a compound of Formula (II-SH)

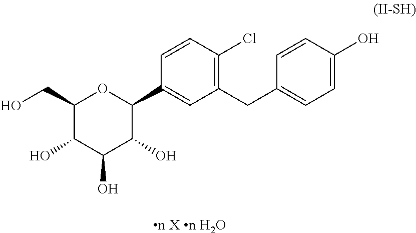

wherein n is 0.5 to 2, X is C₃ to C₆ ketone.

In another aspect there is provided, a compound of general Formula (VI),

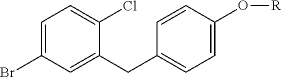

where R is a protecting group selected from trityl, allyl or tetrahydropyran.

In another aspect there is provided, a compound of Formula (IV-P),

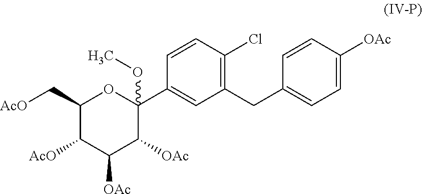 [SMILES: COC1(c2ccc(Cl)c(Cc3ccc(OC(C)=O)cc3)c2)O[C@H](COC(C)=O)[C@@H](OC(C)=O)[C@H](OC(C)=O)[C@H]1OC(C)=O]

In another general aspect, there is provided a co-crystal of empagliflozin and amino acid of Formula (B).

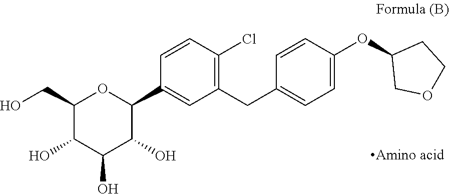 [SMILES: OC[C@H]1O[C@@H](c2ccc(Cl)c(Cc3ccc(O[C@H]4CCOC4)cc3)c2)[C@H](O)[C@@H](O)[C@@H]1O ; "·Amino acid"]

In another aspect, there is provided a process for the preparation of co-crystal of empagliflozin and amino acid, the process comprising:
(a) dissolving empagliflozin and an amino acid in one or more solvents to obtain a reaction mixture;
to (b) optionally warming the reaction mixture to obtain complete dissolution;
(c) cooling the reaction mixture; and
(d) removing the solvent to obtain the co-crystal of empagliflozin and amino acid.

In another aspect, there is provided a process for the preparation of an amorphous form of empagliflozin, the process comprising:
(a) dissolving co-crystal of empagliflozin and L-proline in one or more solvents;
(b) removing the solvents to obtain a residue;
(c) dissolving the residue in another solvent; and
(d) removing the solvent to obtain the amorphous form of empagliflozin.

In another general aspect, there is provided a pharmaceutical composition comprising therapeutically effective amount of co-crystal of empagliflozin and amino acid and one or more pharmaceutically acceptable carriers, excipients or diluents.

In another aspect, there is provided a pharmaceutical composition comprising therapeutically effective amount of amorphous empagliflozin prepared by the process described in the present invention and one or more pharmaceutically acceptable carriers, excipients or diluents.

### BRIEF DESCRIPTION OF THE ACCOMPANYING DRAWINGS

FIG. 1 illustrates a powder X-ray diffraction pattern of the co-crystal of empagliflozin and L-proline.

FIG. 2 illustrates ¹H NMR spectrum of the co-crystal of empagliflozin and L-proline.

FIG. 3 illustrates amorphous form of empagliflozin.

FIG. 4 illustrates the X-ray diffractogram (XRD) of crystalline trityl protected to compound of Formula (VI-a).

FIG. 8 illustrates the differential scanning thermogram (DSC) of crystalline trityl protected compound of Formula (VI-a).

FIG. 6 illustrates a powder X-ray diffraction pattern of hemi methyl ethyl ketone solvate hemihydrate.

FIG. 7 illustrates the TGA of hemi methyl ethyl ketone solvate hemihydrate.

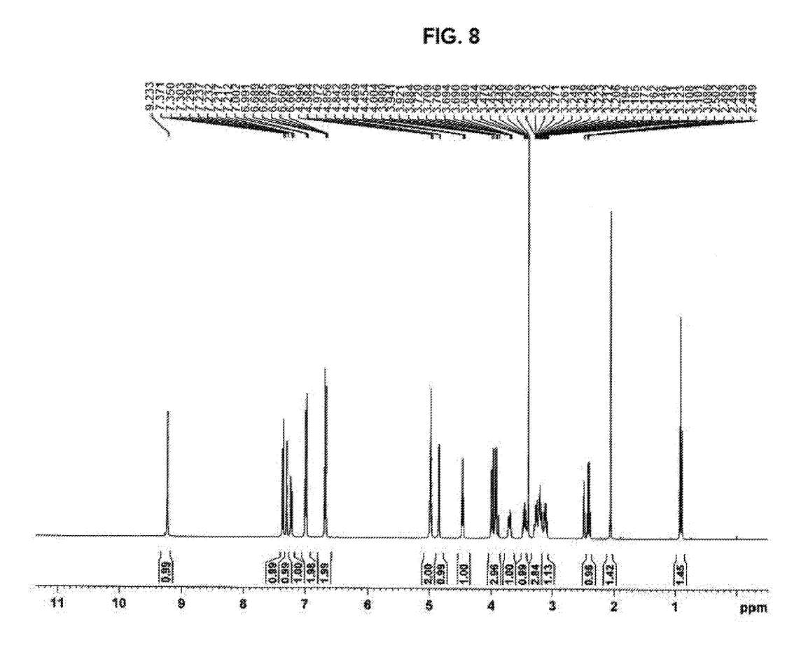

FIG. 8 illustrates the ¹H NMR spectrum of hemi methyl ethyl ketone solvate hemihydrate.

### DETAILED DESCRIPTION OF THE INVENTION

The above and other objects of the present invention are achieved by the process of the present invention, which leads to an improved process for the preparation of amorphous form of empagliflozin.

The terms “protection”, “protecting”, “protected” and “protecting group” refer to the practice of preparing a derivative of a subject compound, wherein one or more functional groups of the compound are prevented from undergoing undesired reactions with a “protecting” functional group.

In one general aspect, there is provided a process for the preparation of empagliflozin, the process comprising:
(a) reacting 5-bromo-2-chlorobenzoic acid of Formula (X) with ethoxy benzene to obtain 5-bromo-2-chloro-4′-ethoxybenzophenone of Formula (IX);

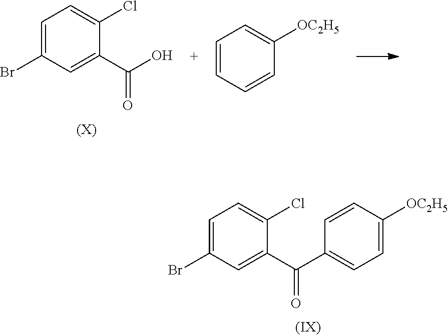

(b) reacting 5-bromo-2-chloro-4′-ethoxybenzophenone of Formula (IX) with reducing reagent to obtain 5-bromo-2-chloro-4′-ethoxydiphenylmethane of Formula (VIII);

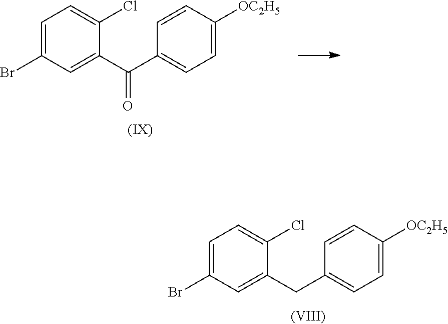

(c) reacting 5-bromo-2-chloro-4′-ethoxydiphenylmethane of Formula (VIII) with Lewis acid to obtain 5-bromo-2-chloro-4′-hydroxydiphenylmethane of Formula (VII);

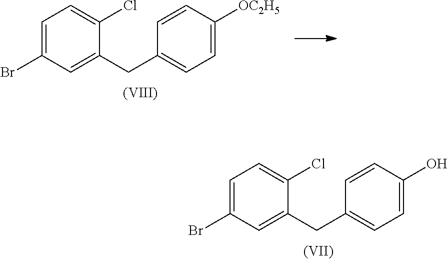

(d) reacting 5-bromo-2-chloro-4′-hydroxydiphenylmethane of Formula (VII) with hydroxy protecting reagent to obtain a compound of Formula (VI);

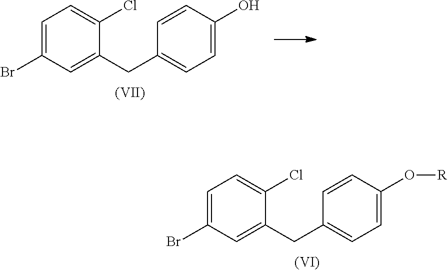

where R is a protecting group selected from trityl, allyl or tetrahydropyran,
(e) reacting the compound of Formula (VI) with a compound of Formula (P-G) to is obtain a compound of Formula (V);

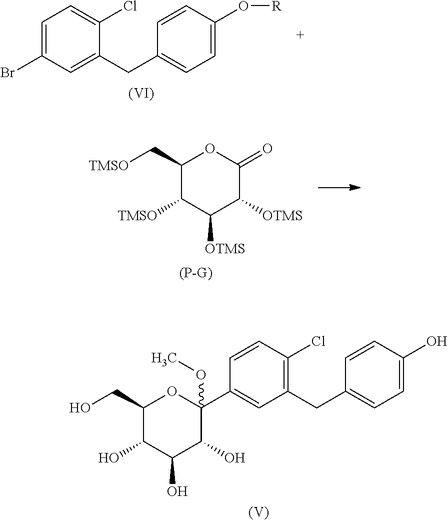

(f) reacting the compound of Formula (V) with a reducing reagent to obtain a compound of Formula (IV);

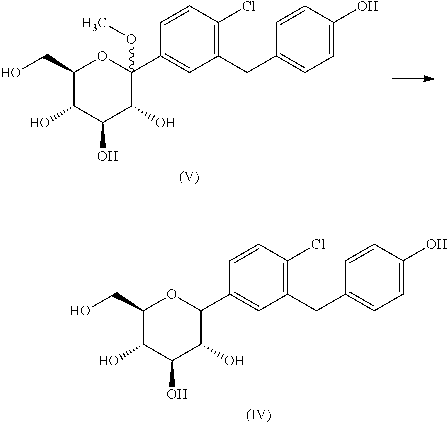 [SMILES: COC1(c2ccc(Cl)c(Cc3ccc(O)cc3)c2)O[C@H](CO)[C@@H](O)[C@H](O)[C@H]1O ; "(V)" ; OC[C@H]1OC(c2ccc(Cl)c(Cc3ccc(O)cc3)c2)[C@H](O)[C@@H](O)[C@@H]1O ; "(IV)"]

(g) reacting the compound of Formula (IV) with a hydroxy protecting reagent to obtain a compound of Formula (III);

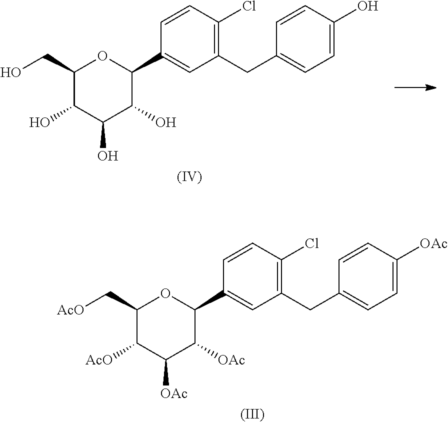

(h) converting the compound of Formula (III) into a compound of Formula (II); and

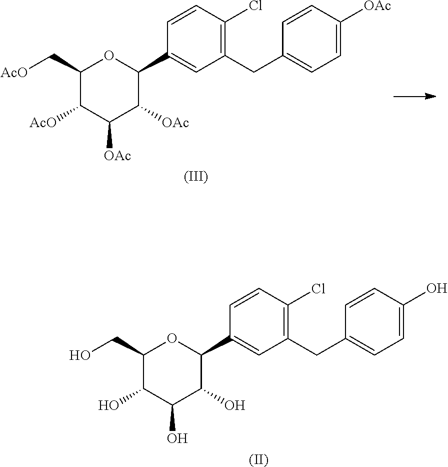

(i) converting the compound of Formula (II) into empagliflozin.

In general, the compound 5-bromo-2-chloro-4′-ethoxybenzophenone of Formula (IX) may be prepared by Friedel-Craft acylation of ethoxy benzene (phenetole) with 5-bromo-2-chlorobenzoyl chloride in suitable solvent such as dichloromethane containing Lewis acid for example AlCl₃ or AlBr₃.

The compound 5-bromo-2-chlorobenzoyl chloride is prepared from 5-bromo-2-chlorobenzoic acid by treatment with oxalyl chloride or thionyl chloride in chlorinated solvent selected from dichloromethane, dichloroethane, carbon tetrachloride, containing a catalytic amount of DMF.

The compound 5-bromo-2-chloro-4′-ethoxydiphenylmethane of Formula (VIII) is prepared by treatment of 5-bromo-2-chloro-4′-ethoxybenzophenone of Formula (IX) with a reducing agent for example Et₃SiH in a solvent for example dichloromethane and acetonitrile or mixture thereof in the presence of a Lewis acid catalyst for example BF₃.Et₂O.

The compound 5-Bromo-2-chloro-4′-ethoxydiphenylmethane of Formula (VIII) may be reacted with various Lewis acids for example BBr₃, BCl₃, AlCl₃, AlBr₃ in the presence of chlorinated solvents for example dichloromethane, dichloroethane, carbon tetrachloride, chloroform to obtain 5-bromo-2-chloro-4′-hydroxy-diphenylmethane of Formula (VII).

The protection of hydroxy group can be carried out by other alternative groups and methods known and well described in the art for example, in “Protective Groups in Organic Synthesis” by Theodora W. Greene, Wiley-Inter science 1981, New York to and references cited therein.

The reaction is carried out in the presence of base, which can be selected from inorganic bases like sodium hydroxide, potassium hydroxide, lithium hydroxide, sodium carbonate, potassium carbonate, lithium carbonate, sodium bicarbonate, potassium bicarbonate, sodium hydride and potassium tert-butoxide or organic base like triethyl amine, diisopropyl amine, diisopropylethylamine, pyridine, morpholine and piperidine. Preferably triethyl amine can be used.

The reaction may be performed in presence of suitable solvent selected from one or more of dichloromethane, dichloroethane, carbon tetrachloride, benzene, toluene, acetonitrile, dimethylformamide, acetone or mixture thereof.

The compound of Formula (P-G) can be prepared by treatment of commercially available D-gluconolactone with a silylating agent for example trimethylsilyl chloride in a solvent such as THF containing a base such as N-methylmorpholine.

The compound of Formula (V) can be prepared, by reaction of n-BuLi or other aryl lithium with the compound of Formula VI and persilylated gluconolactone of Formula (P-G) at lower temperature for example −70 to −75° C.

Subsequently, a methanol solution of a protic acid for example methane sulfonic acid can be reacted to obtain compound of Formula (V).

In general, the solvents for the reaction are one or more of diethyl ether, toluene, methylene chloride, hexane, tetrahydrofuran, dioxane, N-methyl pyrrolidone and mixtures thereof.

The compound of Formula (IV) is prepared by reduction of a compound of Formula (V) with a reducing agent in the presence or absence of Lewis acid. The reducing agents comprises one or more of silanes selected from triethylsilane, tripropylsilane, triisopropylsilane, diphenylsilane, sodium borohydride, sodiumcyano borohydride, zinc borohydride, borane complexes, lithium aluminum hydride or diisobutylaluminum hydride. In particular, triethylsilane may be used. he Lewis acids comprises of boron trifluoride etherate, trimethylsilyl triflate, titanium tetrachloride, tin tetrachloride, scandium triflate, copper (II) triflate or zinc iodide or other acids for example hydrochloric acid, toluenesulfonic acid, trifluoroacetic acid, or acetic acid can also be used.

The reaction may be performed in one or more solvents comprises of methylene dichloromethane, chloroform, diethyl ether, tetrahydrofuran, dioxane, acetonitrile, toluene, hexane, ethanol, water, or mixtures thereof. In particular, the mixture of dichloromethane and acetonitrile may be used.

The reaction may be performed at lower temperature for example −30 to −80° C. The compound of Formula (III) may be prepared by treatment of compound of Formula (IV) with a hydroxy protecting group. The hydroxy protecting group comprises of acetylating agents selected from acetic anhydride, acetyl chloride in one or more solvents comprises of THF, dichloromethane in the presence of diisopropylethylamine and catalyst selected from pyridine or dimethylaminopyridine (DMAP).

The acyl protecting group may be further removed by hydrolysis in an aqueous solvent system comprising one or more of water, isopropanol-water, acetic acid-water, tetrahydrofuran-water or dioxane-water.

The reaction may be performed in the presence of an acid selected from trifluoroacetic acid, hydrochloric acid or sulphuric acid or in the presence of base selected from triethlyamine, ethyldiisopropylamine, sodium carbonate, potassium carbonate, sodium hydroxide, potassium hydroxide or lithium hydroxide. In particular, lithium hydroxide in tetrahydrofuran-water may be used.

The compound of Formula (II) can be reacted with (R)-tetrahydrofuran-3-yl-4-methylbenzenesulfonate to obtain empagliflozin in the presence of base selected from triethlyamine, ethyldiisopropylamine, sodium carbonate, potassium carbonate, cesium carbonate, sodium hydroxide, potassium hydroxide and cesium hydroxide. In particular, cesium carbonate may be used.

In general, the reaction may be performed in one or more of solvent selected from ethanol, isopropanol, butanol, acetone, water, dimethylformamide, dimethylacetamide, N-methyl pyrrolidone, dimethylsulfoxide, tetrahydrofuran, dichloromethane, or mixtures thereof. In particular, dimethylsulfoxide or dimethylformamide may be used.

In general, the reaction may be performed at temperature below 50° C. to control to diastereomer impurity. In particular, the reaction may be performed at 40-50° C.

In another general aspect, there is provided a process for the preparation of empagliflozin, the process comprising:
(a) reacting 5-bromo-2-chloro-4′-ethoxydiphenylmethane of Formula (VIII) with Lewis acid to obtain 5-bromo-2-chloro-4′-hydroxydiphenylmethane of Formula (VII);

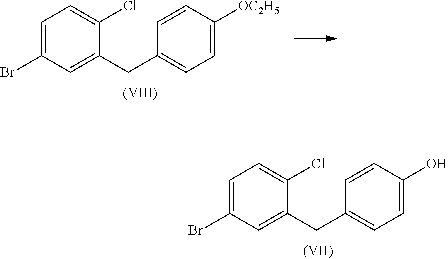

(b) reacting 5-bromo-2-chloro-4′-hydroxydiphenylmethane of Formula (VII) with a hydroxy protecting reagent to obtain a compound of Formula (VI);

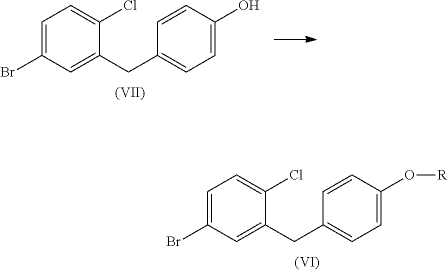

(c) reacting the compound of Formula (VI) with a compound of Formula (P-G) to obtain a compound of Formula (V);

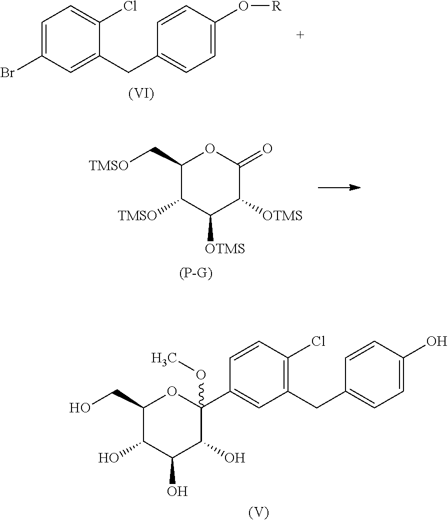

(d) converting the compound of Formula (V) into a compound of Formula (II-SH);

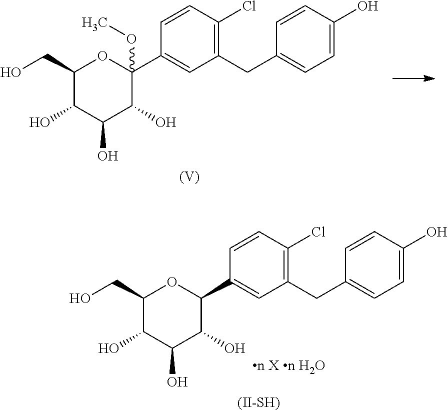

wherein n is 0.5 to 2, X is C₃ to C₆ ketone,
(e) converting the compound of the Formula (II-SH) into a compound of Formula (C); and

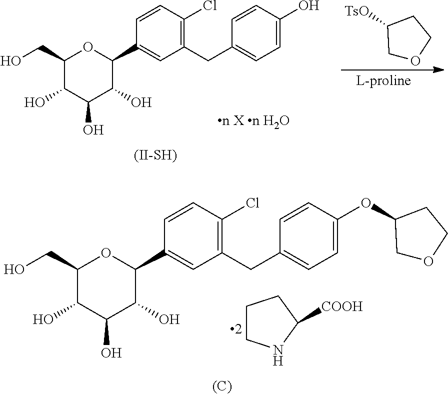

(f) converting the compound of Formula (C) into empagliflozin.

In general, the compounds of Formula (VIII), (VII), (VI) and (V) are prepared as described herein above and herein after the examples.

In general, the compound of Formula (V) may be isolated or may be used directly to in-situ for the next step.

In general, the compound of Formula (II-SH) in step (d) may be particularly a compound wherein n is 0.5 and X is ethyl methyl ketone or acetone.

The compound of Formula (V) is reacted with a reducing agent in the presence or absence of Lewis acid. The reducing agent for the reaction may be selected from triethylsilane, diphenylsilane, tripropylsilane, triisopropylsilane, sodium borohydride or other reducing agents as mentioned herein. In particular, the triethylsilane may be used.

The Lewis acids for the reaction may be selected from boron trifluoride etherate, trimethylsilyl triflate, titanium tetrachloride, tin tetrachloride, hydrochloric acid, toluenesulfonic acid, trifluoroacetic acid and acetic acid may be used. In particular, the boron trifluoride etherate can be used.

The reaction may be performed in one or more solvents selected from methylene dichloromethane, chloroform, diethyl ether, tetrahydrofuran, dioxane, acetonitrile, toluene, hexane, ethanol, water, or mixtures thereof. In particular, the mixture of dichloromethane and acetonitrile is used. The reaction may be performed at a lower temperature −40 to −80° C.

In general, the compound of Formula (II-SH) is further reacted with (R)-tetrahydrofuran-3-yl-4-methylbenzenesulfonate and with an amino acid to obtain empagliflozin amino acid co-crystal.

The reaction may be performed in the presence of a base selected from triethlyamine, ethyldiisopropylamine, sodium carbonate, potassium carbonate, cesium carbonate, sodium hydroxide, potassium hydroxide, and cesium hydroxide. In particular, cesium carbonate may be used.

In general, the solvents for the reaction comprises one or more of dimethylformamide, dimethylacetamide, N-methyl pyrrolidone, dimethylsulfoxide, tetrahydrofuran, dichloromethane, ethanol, isopropanol, butanol, acetone, water, or mixtures thereof. In particular, dimethylsulfoxide dimethylformamide may be used. In general, the reaction may be performed below 50° C. to avoid the formation of diastereomeric impurity and other related impurities. In general, the temperature is 40-50° C.

The compound of Formula (C) is converted to empagliflozin. The reaction may be carried out by dissolving the co-crystal of empagliflozin in one or more solvents and stirring for 30 minutes to 2 hours at ambient temperature. After the completion of the reaction, the solvent may be removed and the residue may be treated with one or more another solvents to obtain empagliflozin.

In general, the solvent for the reaction is selected from water, C₁-C₄-alcohols, tetrahydrofuran, toluene, methylene dichloride, ethylene dichloride, carbon tetrachloride, ethyl acetate, dimethyl sulfoxide, dimethyl formamide, or mixture thereof. In particular, water, ethyl acetate and methanol may be used.

In another general aspect, there is provided a process for the preparation of empagliflozin, the process comprising:
(a) reacting 5-bromo-2-chlorobenzoic acid of Formula (X) with ethoxy benzene to obtain 5-bromo-2-chloro-4′-ethoxybenzophenone of Formula (IX);

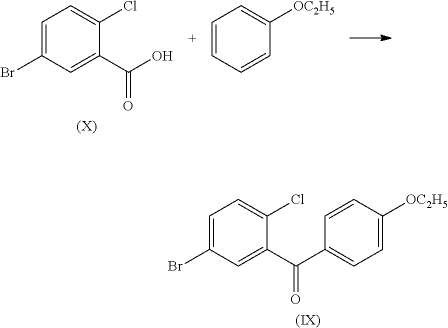

(b) reacting 5-bromo-2-chloro-4′-ethoxybenzophenone of Formula (IX) with reducing reagent to obtain 5-bromo-2-chloro-4′-ethoxydiphenylmethane of Formula (VIII);

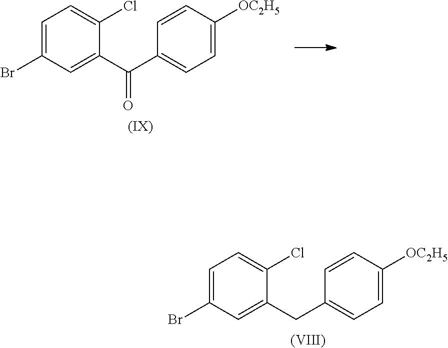

(c) reacting 5-bromo-2-chloro-4′-ethoxydiphenylmethane of Formula (VIII) with Lewis acid to obtain 5-bromo-2-chloro-4′-hydxydroxydiphenylmethane of Formula (VII);

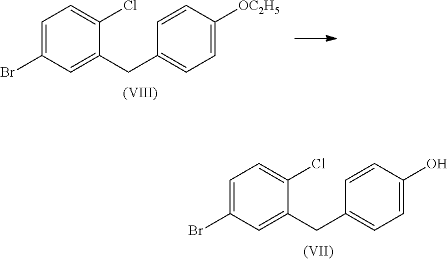

(d) reacting 5-bromo-2-chloro-4′-hydroxydiphenylmethane of Formula (VII) with hydroxy protecting reagent to obtain compound of Formula (VI);

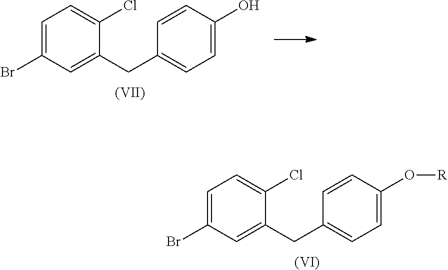

(e) reacting the compound of Formula (VI) with a compound of Formula (P-G) to obtain a compound of Formula (V);

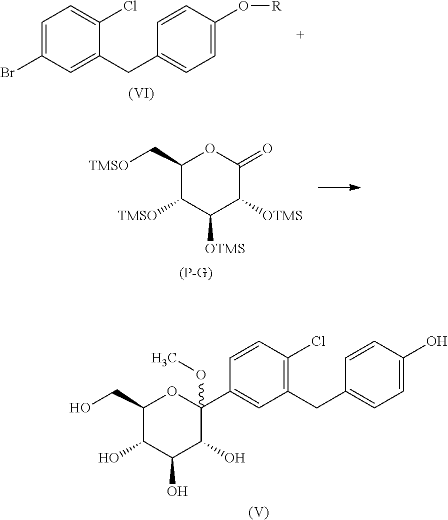

(f) reacting the compound of Formula (V) with hydroxy protecting reagent to obtain a compound of Formula (IV-P);

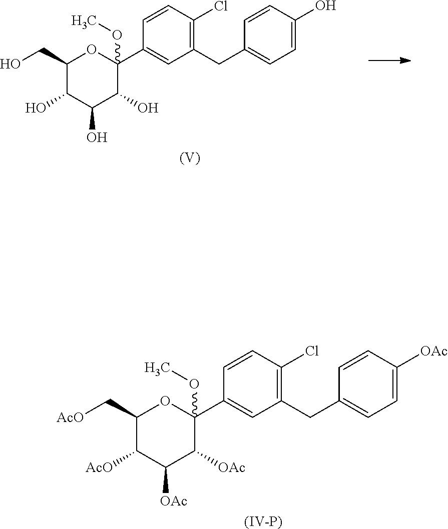 [SMILES: COC1(c2ccc(Cl)c(Cc3ccc(O)cc3)c2)O[C@H](CO)[C@@H](O)[C@H](O)[C@H]1O ; "(V)" ; COC1(c2ccc(Cl)c(Cc3ccc(OC(C)=O)cc3)c2)O[C@H](COC(C)=O)[C@@H](OC(C)=O)[C@H](OC(C)=O)[C@H]1OC(C)=O ; "(IV-P)"]

(g) reacting the compound of Formula (IV-P) with a reducing reagent to obtain compound of Formula (III);

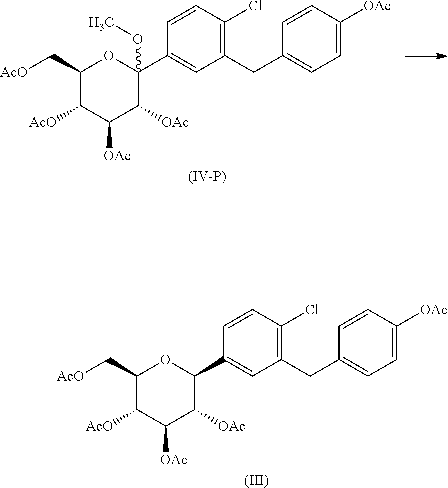

(h) converting the compound of Formula (III) into a compound of Formula (II); and

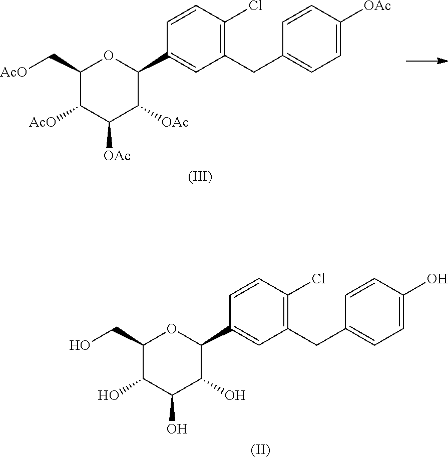

(i) converting compound of Formula (II) into empagliflozin

In general, the compounds of Formula (IX). (VIII), (VII), (VI) and (V) may be prepared as described herein above and herein after in examples.

In general, the compound of Formula (IV-P) may be prepared by treatment of compound of Formula (V) with an acetylating agents selected from acetic anhydride or acetyl chloride in one or more solvents selected from toluene, dichloromethane, hexane, tetrahydrofuran and dioxane, or mixture thereof, in the presence of diisopropyl ethylamine and catalyst selected from pyridine or dimethylaminopyridine (DMAP).

In general, the reducing agent comprises one or more of silanes selected from triethylsilane, tripropylsilane, triisopropylsilane, diphenylsilane, sodium borohydride, sodiumcyano borohydride, zinc borohydride, borane complexes, lithium aluminum hydride or diisobutylaluminum hydride. In particular, triethylsilane may be used.

The reaction may be carried out in the presence or absence of Lewis acid which may be selected from boron trifluoride etherate, trimethylsilyl triflate, titanium tetrachloride, tin tetrachloride, scandium triflate, copper (II) triflate or zinc iodide. In particular, boron trifluoride etherate may be used.

The reaction may be performed in one or more solvents selected from methylene dichloromethane, chloroform, diethyl ether, tetrahydrofuran, dioxane, acetonitrile, toluene, hexane, ethanol, water or mixtures thereof.

The compounds of Formula (III), (II) and (I) are prepared as described herein above and herein below in the examples.

In an another aspect there is provided, a compound of general Formula (VI),

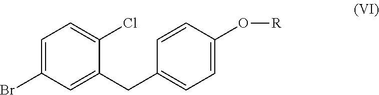

where R is a protecting group selected from trityl, allyl or tetrahydropyran.

In another aspect there is provided, a compound of Formula (VI-a).

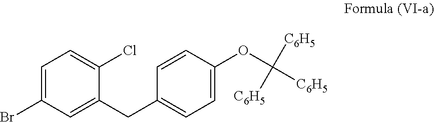

In another aspect, there is provided a crystalline trityl protected compound of Formula (VI-a),

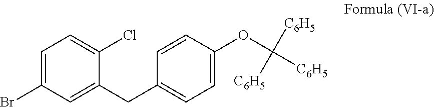

characterized by a powder X-ray diffraction pattern comprising peaks expressed in degrees 2θ at about 5.3, 10.5, 19.8, 21.0, 22.0, 25.0 and 26.3° 2θ±0.2° 2θ.

In general, the crystalline trityl protected compound of Formula (VI-a) may be further characterized by a powder X-ray diffraction pattern comprising peaks expressed in degrees 2θ at about 12.0, 15.9, 16.3, 17.6, 18.2, 20.3, 21.6, 24.0, 24.4, 27.6, 27.9, 29.0, 29.2, 31.7 and 32.2° 2θ±0.2° 2θ and the differential scanning calorimetry having endothermic event around 101.53° C.

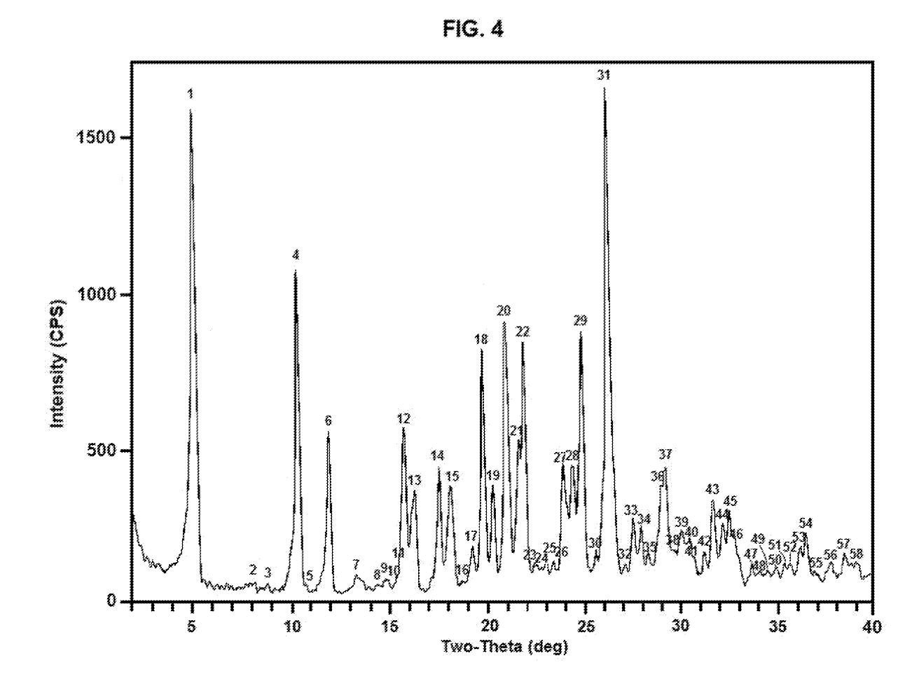

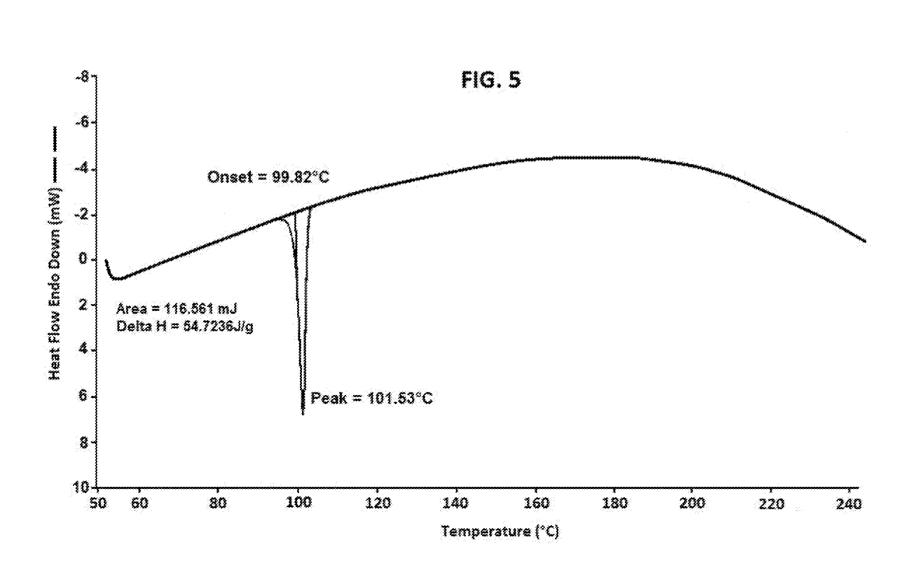

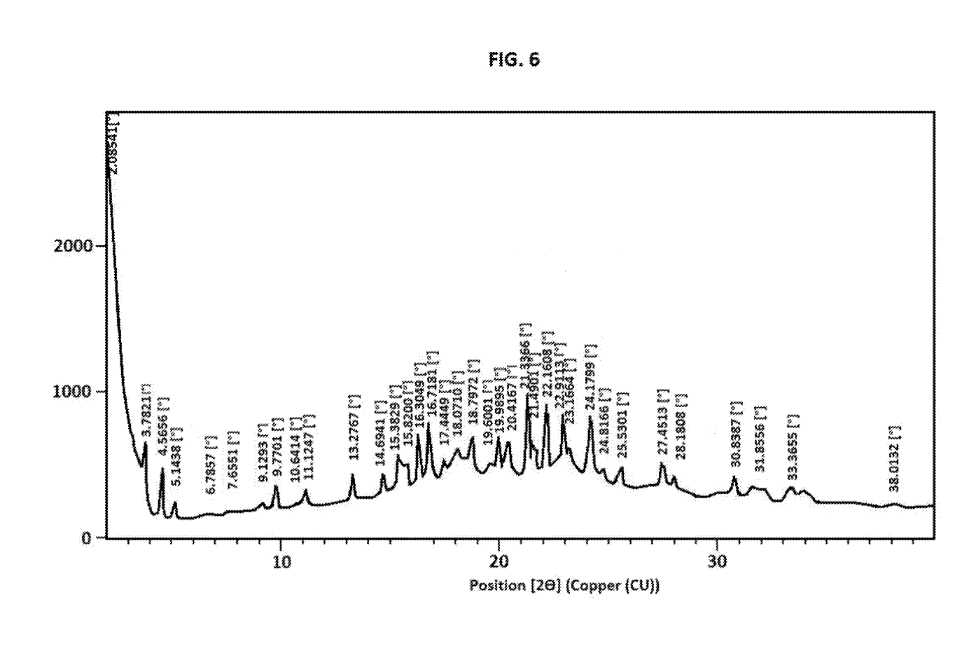

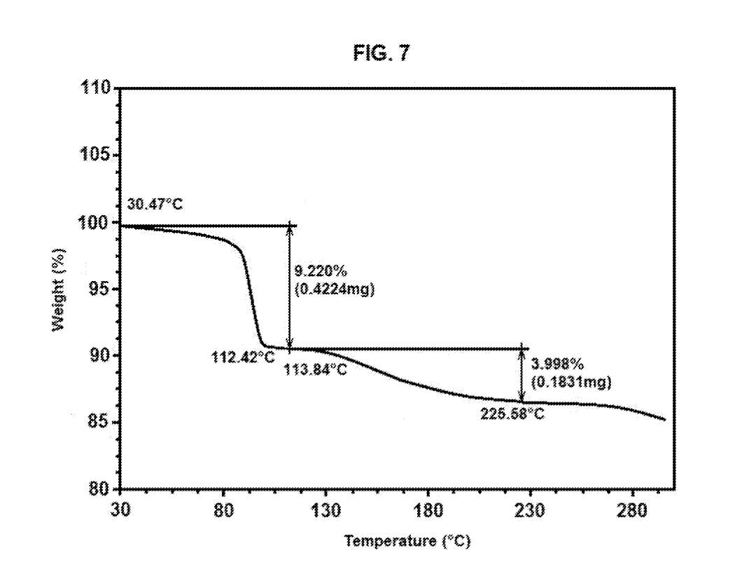

In another general aspect, the crystalline trityl protected compound of Formula (VI-a) is characterized by X-ray powder diffraction substantially as same as depicted in FIG. 4 and differential scanning thermogram (DSC) substantially as same as depicted in FIG. 5.

In another aspect there is provided, a compound of general Formula (IV-P)

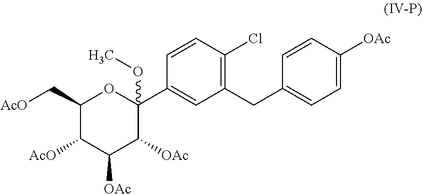

In another general aspect, a compound of general Formula (II-SH),

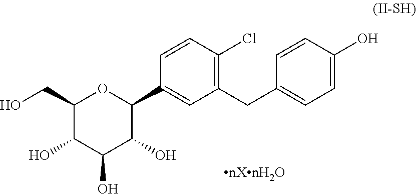

wherein n is 0.5 to 2, X is C₃ to C₆ ketone; particularly n is 0.5 & X is ethyl methyl ketone or acetone.

In another aspect there is provided, a compound of general Formula (II-SH-A),

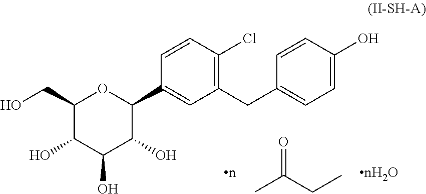

wherein n is 0.5 to 2, particularly n is 0.5.

In general, the compound of Formula (II-SH-A) is a hemi methyl ethyl ketone solvate hemihydrate. The compound is crystalline hemi methyl ethyl ketone solvate hemihydrate characterized by a powder X-ray diffraction pattern comprising peaks expressed in degrees 2θ at about 4.6, 9.8, 16.3, 16.7, 21.3, 22.1, and 24.1° 2θ±0.2°. In general, the crystalline hemi methyl ethyl ketone solvate hemihydrate is further characterized by ¹H NMR (400 MHz, DMSO d6) δ 9.23 (s, 1H), 7.35-7.37 (d, 1H), 7.29-7.30 (d, 1H), 7.21-7.24 (dd, 1H), 6.98-7.00 (d, 2H), 6.66-6.68 (m, 2H), 4.97-4.99 (t, 2H), 4.84-4.85 (d, 1H), 4.45-4.48 (t, 1H), 3.88-4.0 (m, 3H), 3.68-3.72 (m, 1H), 3.42-3.48 (m, 1H), 3.08-3.28 (m, 4H), 2.43-2.49 (q, 1H), 2.1 (s, 1.5H), 0.9 (t, 1.5H).

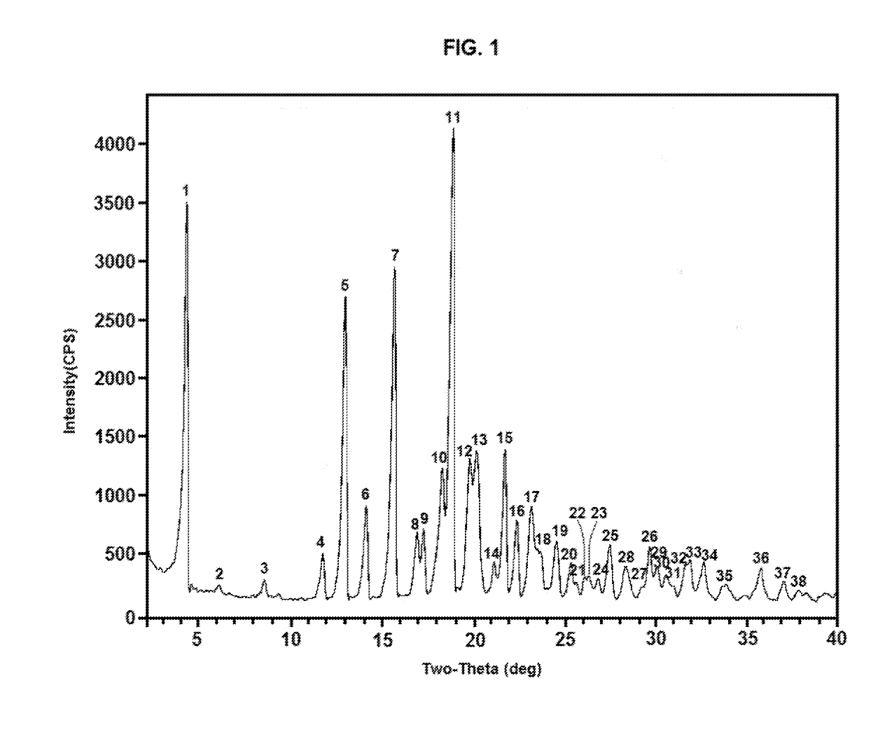

In general, the crystalline hemi methyl ethyl ketone solvate hemihydrate is further to characterized by is characterized by X-ray powder diffraction substantially as same as that depicted in FIG. 6, thermogravimetric analysis (TGA) substantially as same as depicted in FIG. 7 and 1H NMR spectrum substantially as same as depicted in FIG. 9.

In another general aspect, there is provided a co-crystal of empagliflozin and amino acid. The co-crystal of empagliflozin and amino acid can be characterized by its physicochemical parameters, for example those presented hereinafter.

The pharmaceutical co-crystals are crystalline molecular complexes which contain the drug substance along with an additional molecule present in the same crystal structure. The additional molecule or guest has been described in the literature as a co-crystal former. A co-crystal can thus be seen to be a multiple component crystal in which the drug substance and the co-crystal former are arranged in a three dimensional repetitive structure, wherein non-covalent and non-ion pair interactions exist between the drug substance and the co-crystal former, such as hydrogen bonding, pi-stacking, and van der Waals interactions. Co-crystalline forms show different physicochemical properties compared to the drug substance alone, including melting point, chemical reactivity, apparent solubility, dissolution rate, optical and mechanical properties, vapor pressure, and density. These properties can have a direct effect on the ability to process and/or manufacture a drug substance and the corresponding finalized dosage forms, as well as an effect on drug product stability, dissolution, and bioavailability.

In another general aspect, there is provided a process for the preparation of co-crystal of empagliflozin and amino acid, the process comprising:
(a) dissolving empagliflozin and an amino acid in one or more solvents to obtain the reaction mixture;
(b) optionally warming the reaction mixture to complete dissolution;
(c) cooling the reaction mixture; and
(d) removing the solvent to obtain the co-crystal of empagliflozin and amino acid. Empagliflozin can be prepared by any of the method described in the literature mentioned herein above.

In general, the formation of co-crystals of empagliflozin and amino acid can be carried out by using amino acids selected from glycine, L-proline, L-threonine, L-cysteine, L-methionine, L-phenylalanine, L-tyrosine, L-asparagine, L-aspartic acid, L-glutamine, L-glutamic acid, L-lysine, L-arginine, L-histidine, L-serine, L-tryptophan, L-alanine, L-valine, L-leusine, L-isoleusine, D-asparagine, D-aspartic acid, D-glutamine, D-phenylalanine, D-alanine, D-valine, D-leusine, D-glutamic acid, D-arginine, D-serine, D-threonine, D-methionine, D-isoleusine and D-proline. In general during the process, empagliflozin and amino acid can be dissolved in one or more solvents. Both the ingredients may be dissolved in the same solvent, either together or separately in a different solvent. In case of separate dissolution of both ingredients, the two solutions are mixed.

Empagliflozin can be dissolved in one or more solvents whereupon amino acid is added. The whole reaction mixture is warmed until complete dissolution is observed. The solution is allowed to cool back to room temperature or much lower temperatures as the co-crystal precipitates or antisolvent may be added for the crystallization. The co-crystal thus formed can be filtered off to isolate and optionally washed and dried.

In general, the solvents can be selected from one or more of water, dimethyl formamide, dimethyl sulfoxide, dimethylacetamide, N-methyl pyrrolidone or ethanol or toluene or mixture thereof.

In another general aspect, there is provided a co-crystal of empagliflozin and L-proline of Formula (C),

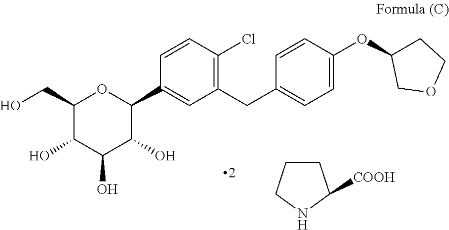 [SMILES: OC[C@H]1O[C@@H](c2ccc(Cl)c(Cc3ccc(O[C@H]4CCOC4)cc3)c2)[C@H](O)[C@@H](O)[C@@H]1O ; O=C(O)[C@@H]1CCCN1]

The co-crystal of empagliflozin and L-proline was found to be 1:2 by the analysis described herein below.

In another aspect, there is provided a process for the preparation of co-crystal of empagliflozin and L-proline of Formula (C),

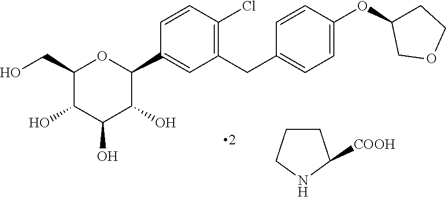 [SMILES: OC[C@H]1O[C@@H](c2ccc(Cl)c(Cc3ccc(O[C@H]4CCOC4)cc3)c2)[C@H](O)[C@@H](O)[C@@H]1O ; O=C(O)[C@@H]1CCCN1]

the process comprising:
(a) dissolving empagliflozin and L-proline in one or more solvents to obtain a reaction mixture;
(b) optionally warming the reaction mixture to obtain complete dissolution;
(c) cooling the reaction mixture; and
(d) removing the solvent to obtain the co-crystal of empagliflozin and L-proline.

In general during the process, empagliflozin and L-proline can be dissolved in one or more solvents. Both the ingredients may be dissolved in the same solvent, either together or separately in a different solvent. In case of separate dissolution of both ingredients, the two solutions are mixed.

Empagliflozin can be dissolved in one or more solvents whereupon L-proline is added. The reaction mixture is warmed until complete dissolution is observed. The solution is allowed to cool to lower temperatures as the co-crystal precipitates or antisolvent may be added for the crystallization. The co-crystal thus formed can be filtered off to isolate and optionally washed and dried.

In general, the solvents comprise one or more of water, dimethyl formamide, dimethyl sulfoxide, dimethylacetamide, N-methyl pyrrolidone or ethanol or toluene or mixture thereof. In particular, ethanol, toluene or mixture thereof.

Co-crystalline forms of a drug substance are characterized by a number of methods including, for example, X-ray powder diffraction, microscopy, thermal analysis (e.g. differential scanning calorimetry, thermal gravimetric analysis and hot-stage microscopy), spectroscopy (e.g., infrared (IR) and near infrared (NIR), Raman, solid-state nuclear magnetic resonance (SS NMR)), and in particular by single crystal X-ray diffraction.

The co-crystal of empagliflozin and L-proline can be characterized by a powder X-ray diffraction pattern comprising peaks expressed in degrees 2-theta at about 4.3°, 12.9°, 15.6°, 18.7°, 20.0° and 21.6° 2θ±0.2° 2θ.

The co-crystal of empagliflozin and L-proline can be further characterized by a powder X-ray diffraction pattern comprising peaks expressed in degrees 2-theta at about 11.7°, 14.0°, 16.90, 17.2°, 18.2°, 19.7°, 22.2°, 23.1°, 24.4°, 27.4° and 29.6° 2θ±0.2° 2θ.

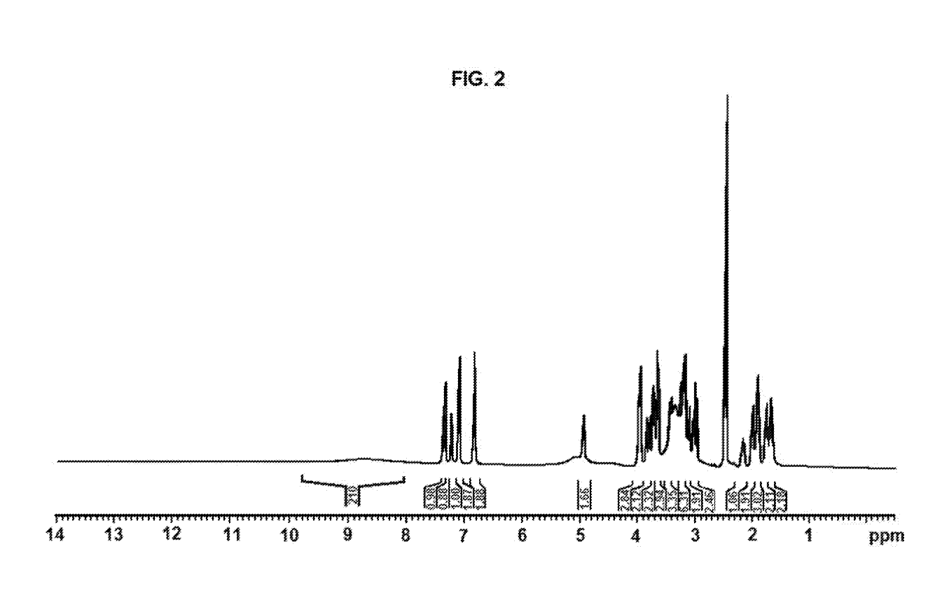

In an another aspect, there is provided co-crystal of empagliflozin and L-proline characterized by X-ray powder diffraction pattern substantially as same as depicted in FIG. 1 and ¹H NMR spectrum substantially as same as depicted in FIG. 2.

The X-ray powder diffraction spectrum was measured using X-Ray Diffractometer, D/Max-2200/PC Make or PANalytical Make or equivalent and having CuKα source.

The ¹H NMR spectrum was measured by Broker 400 MHz spectrometer wherein samples were dissolved in DMSO-d6 for analysis.

In another general aspect, there is provided a process for the preparation of an amorphous form of empagliflozin, the process comprising:
(a) dissolving co-crystal of empagliflozin and L-proline in one or more solvents;
(b) removing the solvents to obtain a residue;
(c) dissolving the residue in one or more another solvent; and
(d) removing the solvent to obtain the amorphous form of empagliflozin.

In general, the solvent in step (a) and (c) comprises one or more of water, C₁-C₄-alcohols, tetrahydrofuran, toluene, methylene dichloride, ethylene dichloride, carbon tetrachloride, ethyl acetate, dimethyl formamide, dimethyl sulfoxide, or mixture thereof. In particular, water, tetrahydrofuran, ethyl acetate, methanol can be used.

The solvent may be removed by one or more techniques selected from a rotational distillation device such as a Buchi Rota vapor, spray drying, agitated thin film drying (“ATFD”) or freeze drying (lyophilization) or any other suitable technique. The solvent can be removed by spray drying a solution of empagliflozin and that to involves the spray drying of feed stock, which is prepared as discussed below. The feedstock is dozed into the spray-drying instrument LU-222 Advanced Model Spray dryer and spray drying is carried out under the following parameters.

| Sr. no | Parameters | Conditions | |
|---|---|---|---|
| A | Feed pump flow rate | 3 | ml/min |
| B | Inlet temperature | 35°-80° | C. |
| C | Outlet temperature | 30°-75° | C. |
| D | Aspirator flow rate | 100 | Nm³/hr |
| E | Vacuum | 100-160 | mmWC |
| F | Atomization pressure | 0.80-0.90 | Kg/cm² |

In the present invention, feed stock of empagliflozin can be prepared by dissolving empagliflozin in the solvent selected from acetone, C₁-C₄ alcohol, C₂-C₆-acetate, acetonitrile, methylene dichloride, water or mixture thereof. Preferably acetone, methanol, ethanol, ethyl acetate or mixtures thereof can be used.

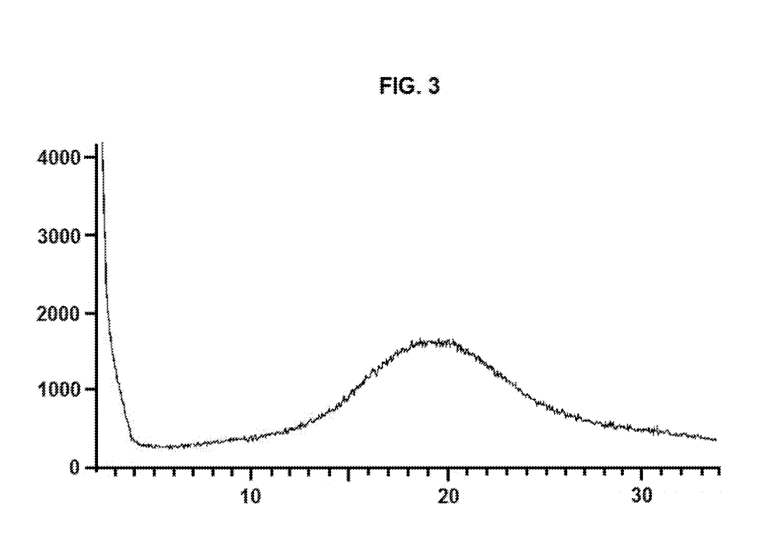

In another general aspect, the amorphous form of empagliflozin is characterized by X-ray powder diffraction pattern substantially as same as depicted in FIG. 3.

In another general aspect, there is provided a process for the preparation of empagliflozin, according to the reaction scheme-I substantially as depicted herein after.

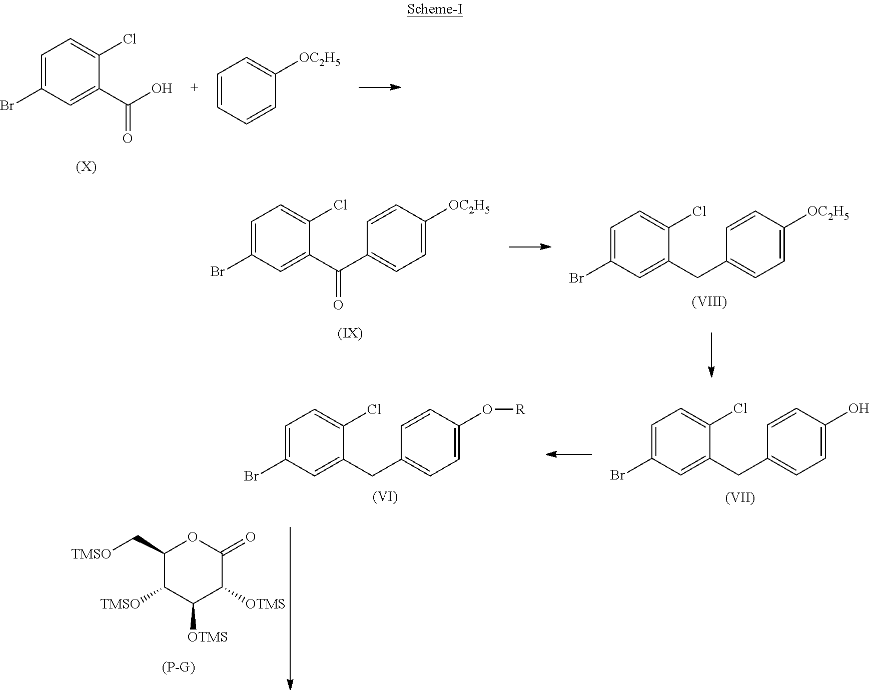

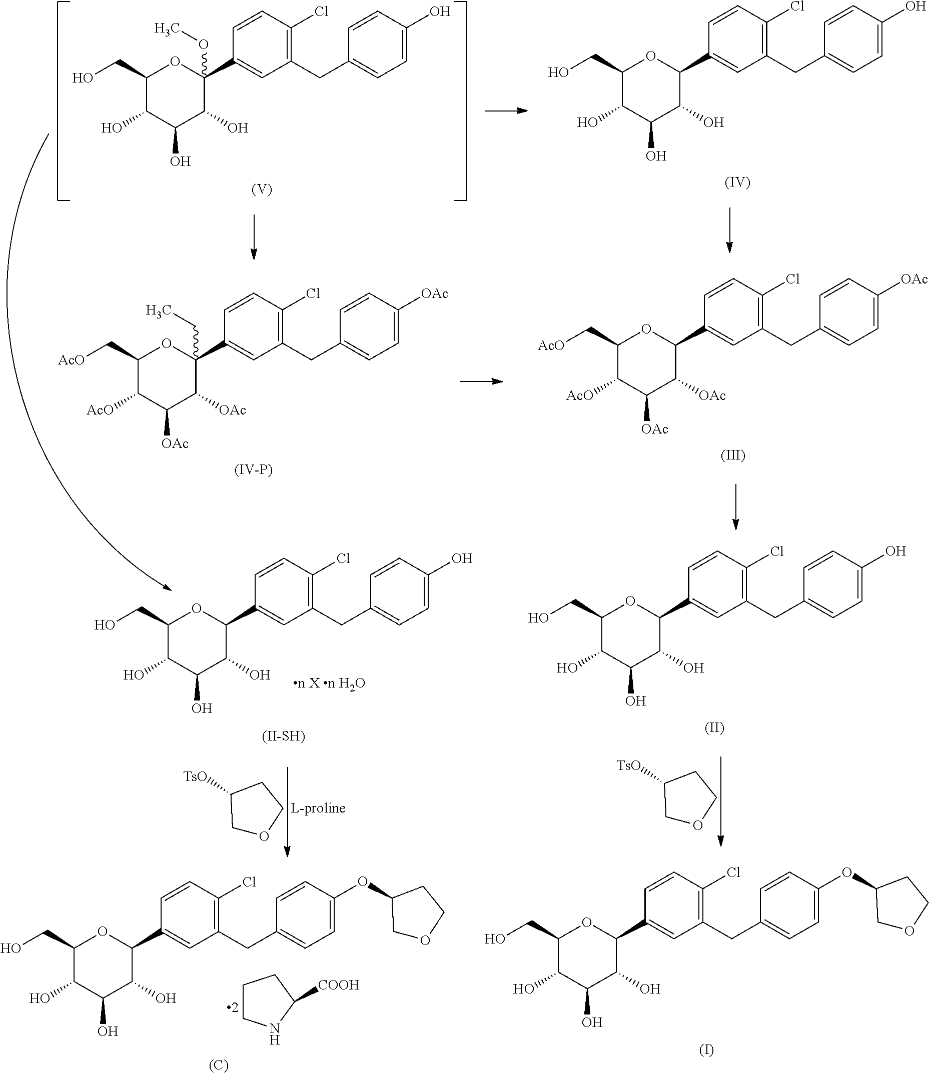

In another general aspect, there is provided empagliflozin substantially free from one or more of impurities as determined by area percentage of HPLC, represented by structural Formula as herein below:

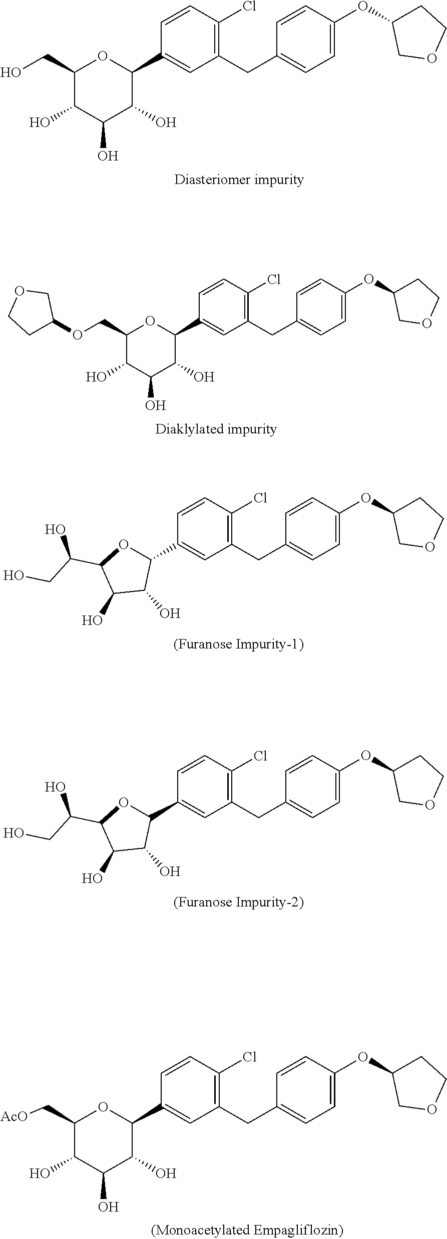

In another general aspect, there is provided a pharmaceutical composition comprising empagliflozin substantially free from one or more of impurities and pharmaceutically acceptable excipients, diluents or carriers.

In another aspect, there is provided a pharmaceutical composition comprising therapeutically effective amount of co-crystal of empagliflozin and amino acid and one or more pharmaceutically acceptable excipients for the treatment of diabetes. In another aspect, there is provided a pharmaceutical composition comprising therapeutically effective amount of amorphous empagliflozin and one or more pharmaceutically acceptable excipients for the treatment of diabetes.

The pharmaceutically acceptable excipients may include surfactants, solubilizers, disintegrants such as microcrystalline cellulose, starch, sodium starch glycolate, and crosslinked carboxy methyl cellulose sodium, crosslinked PVP, pigments, flavors, fillers, lubricants, glidants, preservatives, thickening agents, buffering agents, and pH modifiers.

The invention is explained in its best way by examples as given herein below. It will be apparent to those skilled in the art that the various modifications and variations can be made in the present invention and specific examples provided herein without departing from the spirit or scope of the invention.

### EXAMPLES

### Example-1: Preparation of 5-bromo-2-chloro-4′-ethoxybenzophenone of Formula (IX)

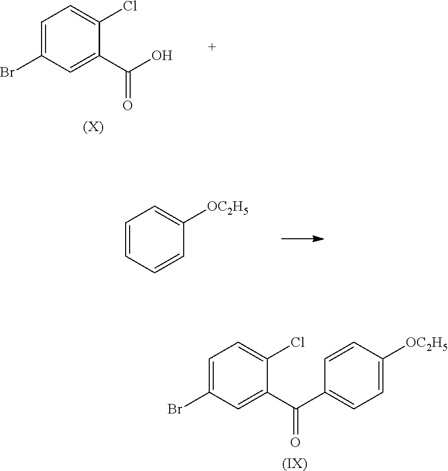

In a round bottom flask, 2-chloro-5-bromobenzoic acid (100 gm), dichloro methane (500 ml) and 0.5 mL DMF were taken under nitrogen atmosphere. The reaction mixture was stirred for 15-20 minutes at 25-30° C. and then cooled to 10-15° C. and slowly thionyl chloride (101 gm) was added. The reaction mixture was stirred for 15-20 minutes at 10-15° C. and then stirred at 40-45° C. for 5-6 hours. The solvent was distilled out under vacuum. 250 mL dichloro methane and 62.3 g Anhydrous AlCl₃ were taken in another round bottom flask and cooled to 0-5° C. and then 54.6 gm Phenetole was added and stirred for 15-20 minutes at 0-5° C. The above prepared acid chloride mixture was added into the reaction mixture under nitrogen atmosphere at 0-5° C. and stirred at this temperature for an hour. After completion of the reaction, the reaction mixture was poured into HCl solution, and organic layer and aqueous layers were separated. Organic layer was washed with water and the organic solvent was distilled out under vacuum. 200 ml ethanol was added and stirred for 15-20 minutes at 40-45° C. and then the reaction mixture was stirred at 0-5° C. for an hour. The reaction mass then filtered and washed with pre-cooled ethanol to obtain 5-Bromo-2-chloro-4′-ethoxybenzophenone of Formula (IX).

### Example-2: Preparation of 5-bromo-2-chloro-4′-ethoxydiphenylmethane of Formula (VIII)

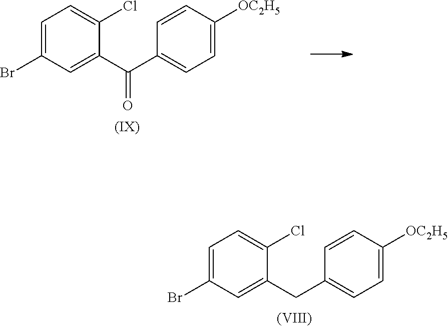

In a round bottom flask, 95 gm benzophenone of Formula (IX), 950 mL acetonitrile were taken and reaction mass was stirred at 25° C. to 35° C. for 15-20 minutes. The reaction mixture was cooled to 10° C. to 15° C. and 130 gm of triethyl silane was slowly added. The reaction mass was stirred at 10° C. to 15° C. for 30-40 minutes and then 159 gm BF₃-etharate was slowly added into the reaction mixture at 10° C. to 15° C. and stirred for 15-20 minutes. The reaction mixture was stirred at 40° C. to 45° C. for 2 hours. After completion of the reaction, 380 mL toluene and 380 ml sat.NaHCO₃ solution were into the reaction mixture. The reaction mixture was stirred at 25° C. to 35° C. for 15-20 minutes. The organic layer and aqueous layers were separated. The separated organic layer was washed with water. The organic solvent was removed under vacuum. 200 mL isopropyl alcohol was added into the reaction mass and stirred at 45° C. to 50′C for 30 minutes. The reaction mixture was cooled to 0° C. to 5° C. and stirred foe 1-2 hours. The product thus obtained was filtered and washed with pre-cooled isopropyl alcohol and the dried under vacuum oven to obtain 5-Bromo-2-chloro-4′-ethoxydiphenylmethane of Formula (VIII).

### Example-3: Preparation of 5-bromo-2-chloro-4′-hydroxydiphenylmethane of Formula (VII)

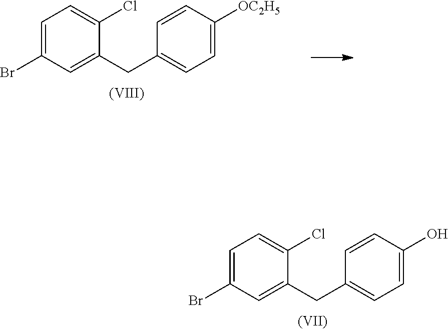

In a round bottom flask, 150 g 5-bromo-2-chloro-4′-ethoxydiphenylmethane and 600 mL dichloro methane were taken and reaction mass was stirred at 25° C. to 35° C. for 15-20 minutes. The reaction mixture was cooled to 0° C. to 5° C. and 86.5 gm boron tribromide was slowly added at 0° C. to 5° C. and the reaction mixture was stirred at this temperature for 3-4 hours. After completion of the reaction, potassium carbonate solution was added to adjust pH 8-9 at 0° C. to 5° C. and then HC solution was added slowly to adjust pH 1-2 at 0° C. to 5° C. The reaction mixture was stirred for 30 minutes at this temperature. The organic and aqueous layers were separated and the organic layer was washed with water. The organic solvent was distilled out under vacuum. 600 mL cyclohexane was added into the reaction mixture and heated to 75° C. to 85° C. to get a clear solution. The reaction mixture was slowly cooled to 25° C. to 35° C. and stirred for half an hour and then stirred for one hour at 0° C. to 5° C. The product was filtered and washed with pre-cooled cyclohexane and dried to obtain 5-Bromo-2-chloro-4′-hydroxydiphenylmethane of Formula (VII).

### Example-4: Preparation of Compound of Formula (VI)

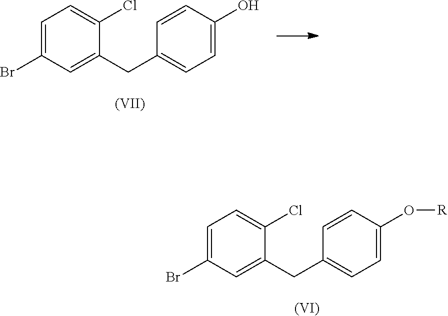

(Wherein R is trityl group)

In a round bottom flask, 150 g 5-bromo-2-chloro-4′-hydroxydiphenylmethane and 300 mL dichloro methane were taken and stirred for 15-20 minutes at 25° C. to 35° C. The reaction mixture was cooled to 0° C. to 5° C. and 34 gm triethyl amine was added and then trityl chloride solution (51.5 g trityl chloride dissolve in 150 mL dichloro methane) was added into the reaction mixture. The reaction mixture was stirred 0° C. to 5° C. for 2-3 hours. After completion of the reaction, 200 mL water was added. The organic and aqueous layers were separated. Organic layer was washed with water. The organic solvent was removed under vacuum. 250 mL methyl-ter-butyl ether was added and at 45° C. to 500° C. then cooled to 0° C. to 5° C. and stirred at 0° C. to 5° C. for an hour. The product was filtered and washed with pre-cooled methyl-ter-butyl ether and dried to obtain trityl protected compound of Formula (VI).

### Example-5: Preparation of Compound of Formula (V)

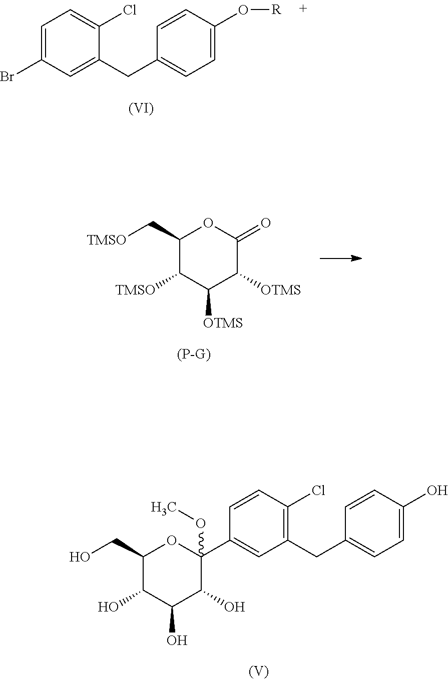

In a round bottom flask, 750 mL of THF and 50 g of trityl-protected compound of Formula (VI) were taken under nitrogen atmosphere and stirred 25° C. to 35° C. for 10-15 minutes. 45.3 g TMS gluconolactone of Formula (P-G) was added and the reaction mixture was cooled to −70° C. to −75° C. 78 mL of 23% n-BuLi was added slowly at −70° C. to −75° C. and stirred for 1-2 hours. After completion of the reaction, 26.8 gm methane sulfonic acid and 150 mL methanol were added into the reaction mixture at −70° C. to −75° C. and stirred for 12.0 h at 25° C. to 35° C. then saturated to sodium bicarbonate solution was added to adjust pH 6-7 and the reaction mixture was stirred for 30 minutes at 25° C. to 35° C. The solvent was removed under vacuum. 500 mL ethyl acetate was added into the residue and stirred for 15 minutes. The organic layer and aqueous were separated and the organic solvent was distilled out under vacuum. 150 mL acetonitrile was added into the residue and stirred for is 15 min at 25° C. to 35° C. 150 mL cyclohexane was added into the reaction mixture and stirred for 15 min. Layers were separated and acetonitrile layer and washed two times with 150 mL cyclohexane. The solvent was removed under vacuum to obtain compound of Formula (V).

### Example-6: Preparation of Compound of Formula (IV)

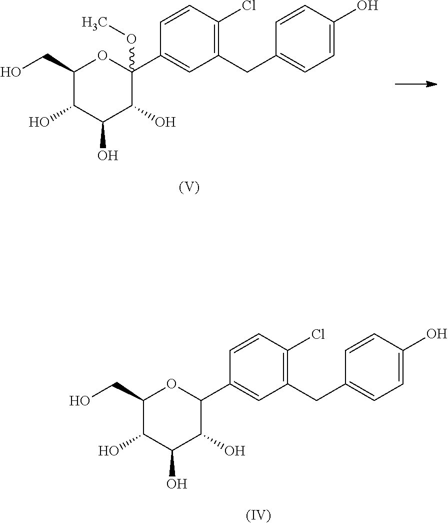 [SMILES: COC1(c2ccc(Cl)c(Cc3ccc(O)cc3)c2)O[C@H](CO)[C@@H](O)[C@H](O)[C@H]1O ; "(V)" ; OC[C@H]1OC(c2ccc(Cl)c(Cc3ccc(O)cc3)c2)[C@H](O)[C@@H](O)[C@@H]1O ; "(IV)"]

In a round bottom flask 105 mL of dichloromethane, 21 g of compound of Formula (V) and 105 mL acetonitrile were taken under nitrogen atmosphere and stirred for 15-20 minutes. 17.90 gm triethyl silane was added and stirred at 25° C. to 35° C. for half an hour. The reaction mixture was cooled to −35° C. to −40° C. and 21.77 gm. BF₃-etherate was added slowly and the reaction mixture was stirred for half an hour at −35° C. to −40° C. The reaction mixture then stirred for 3-4 hours at −5° C. to −10° C. After completion of the reaction, saturated sodium bicarbonate solution was added into the reaction mixture to adjust pH 6-7 at −5° C. to −10° C. The layers were separated. The solvent was distilled out under vacuum from the organic layer. 1050 mL ethyl acetate was added into the reaction mixture and stirred for 15 min at 25° C. to 35° C. The reaction mixture was washed with 100 mL saturated NaCl solution and then the layers were separated. The solvent from the organic layer was distilled out under vacuum to obtain compound of Formula (IV).

### Example-7: Preparation of Compound of Formula (HI)

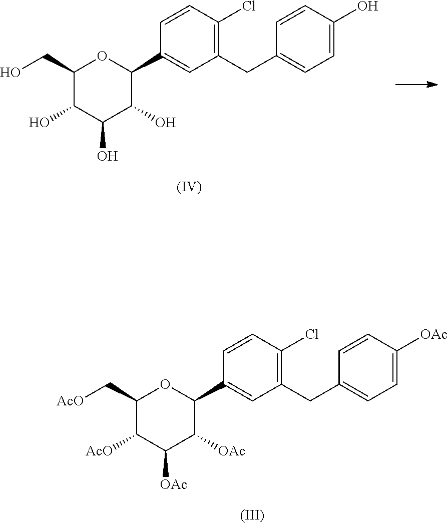 [SMILES: OC[C@H]1O[C@@H](c2ccc(Cl)c(Cc3ccc(O)cc3)c2)[C@H](O)[C@@H](O)[C@@H]1O ; "(IV)" ; CC(=O)OC[C@H]1O[C@@H](c2ccc(Cl)c(Cc3ccc(OC(C)=O)cc3)c2)[C@H](OC(C)=O)[C@@H](OC(C)=O)[C@@H]1OC(C)=O ; "(III)"]

In a round bottom flask, 8 mL THF and 8 gm compound of Formula (IV) were taken at 25° C. to 35° C. 21.7 gm diisopropyl ethylamine and 0.8 gm 4-dimethylaminopyridine were added into the reaction mixture at 25° C. to 35° C. The reaction mixture was cooled to 0° C. to 5° C. and 12.86 gm acetic anhydride was is added and stirred for 3-4 hours at 0° C. to 5° C. After completion of the reaction, the solvent was removed under vacuum and 100 mL dichloromethane and 100 ml water were added and the reaction mixture was stirred for 15-20 minutes. The organic and aqueous layers were separated. The organic layer was washed with 10% Phosphoric acid solution and then washed with saturated sodium bicarbonate solution. The solvent from the organic layer was distilled out under vacuum. 20 mL methanol was added into the reaction mixture and heated at 65° C. to 70° C. to get clear solution then that was cooled and stirred for an hour at 0° C. to 5° C. The product was filtered and washed with pre-cooled methanol to obtain compound of Formula (III).

### Example-8: Preparation of Compound of Formula (II)

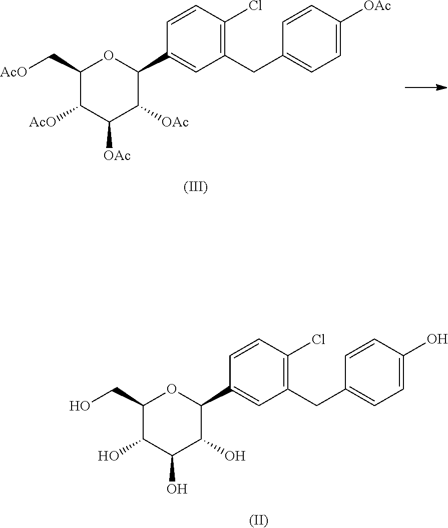 [SMILES: CC(=O)OC[C@H]1O[C@@H](c2ccc(Cl)c(Cc3ccc(OC(C)=O)cc3)c2)[C@H](OC(C)=O)[C@@H](OC(C)=O)[C@@H]1OC(C)=O ; "(III)" ; OC[C@H]1O[C@@H](c2ccc(Cl)c(Cc3ccc(O)cc3)c2)[C@H](O)[C@@H](O)[C@@H]1O ; "(II)"]

In a round bottom flask, 30.8 mL methanol, 3.5 gm compound of Formula (III), 19.25 ml THF and 10.25 ml water were taken and stirred for 15-20 minutes at 25° C. to 35° C. The reaction mixture was cooled to 15° C. to 20° C. 0.29 gm lithium hydroxide and 2 mL water were added into the reaction mixture and stirred for an hour. After completion of the reaction, 40 mL water and 40 mL ethyl acetate were added and stirred for half an hour. The organic and aqueous layers were separated. Organic layer was dried over sodium sulfate. The solvent was distilled out under to vacuum to obtain compound of Formula (II).

### Example-9: Preparation of Compound of Formula (I)

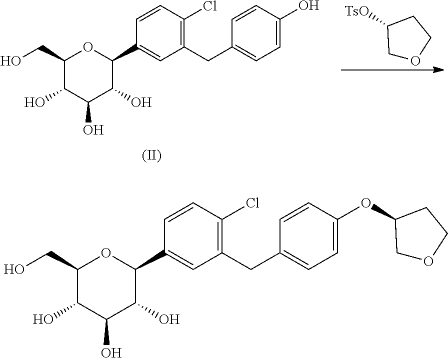 [SMILES: OC[C@H]1O[C@@H](c2ccc(Cl)c(Cc3ccc(O)cc3)c2)[C@H](O)[C@@H](O)[C@@H]1O ; Cc1ccc(S(=O)(=O)O[C@@H]2CCOC2)cc1 ; "(II)" ; OC[C@H]1O[C@@H](c2ccc(Cl)c(Cc3ccc(O[C@H]4CCOC4)cc3)c2)[C@H](O)[C@@H](O)[C@@H]1O]

In a round bottom flask, 1.2 g compound of Formula (II), 1.2 g cesium carbonate, 0.81 (R)-tetrahydrofuran-3-yl-4-methylbenzenesulfonate and 18 mL DMF or is DMSO were added at 25° C. to 35° C. The reaction mixture was heated to 35 to 40° C. and stirred for 24-36 hrs. The reaction mixture then cooled to 25° C. to 35° C. 40 mL water and 20 ml toluene were added stirred for 15-20 minutes. Layers were separated. The aqueous layer was extracted with dichloromethane, dried over sodium sulfate. The solvent was distilled out under vacuum. 10 mL ethanol was added and stirred for 30 minutes. The reaction mixture was stirred at 0-5° C. for an hour. The product was filtered and washed with pre-cooled ethanol and dried to obtain empagliflozin.

### Example-10: Preparation of Compound of Formula (II-SH-A)

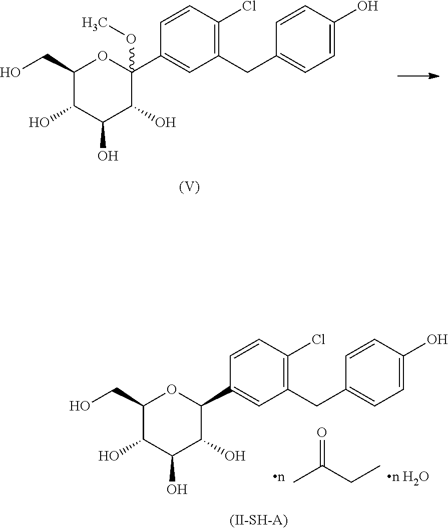

where n is 0.5

In a round bottom flask, 100 g compound of Formula (V), 1000 mL acetonitrile and 1000 mL methylene dichloride were taken and stirred at 25-35° C. 84.92 g triethylsilane was added at 25 to 35° C. The reaction mixture was cooled to −60 to −70° C. 103.65 g boron trifluoride etherate was added using addition funnel in 2-3 hours at −60 to −70° C. and stirred for one hour. The temperature was raised to −10 to 0° C. The reaction mixture was stirred at 0 to −10° C. for 4-5 hours. Alter completion to of the reaction, the pH was adjusted to 7 using sodium bicarbonate solution. The reaction mixture was stirred at 25 to 35° C. for 30-40 minutes. The layers were separated The aqueous layer was washed two-three times with ethyl acetate. The organic layers was combined and dried over sodium sulphate. The solvent was removed by distillation under vacuum. 100 mL ethyl methyl ketone was added and distilled out under vacuum at 40-50° C. then again 800 mL ethyl methyl ketone was added and the reaction mixture was heated to 75-80° C. The reaction mixture was cooled to 10-20° C. and stirred for 2 hours. The solid was filtered and washed with 100 mL ethyl methyl ketone at 25-30<C and then dried under vacuum to obtain the Formula (II-SH-A). M.P.: 87.3-91° C. ¹H NMR (400 MHz, DMSO d6) δ 9.23 (s, 1H), 7.35-7.37 (d, 1H), 7.29-7.30 (d, 1H), 7.21-7.24 (dd, 1H), 6.98-7.00 (d, 2H), 6.66-6.68 (m, 2H), 4.97-4.99 (t, 2H), 4.84-4.85 (d, 1H), 4.45-4.48 (t, 1H), 3.88-4.0 (m, 3H), 3.68-3.72 (m, 1H), 3.42-3.48 (m, 1H), 3.08-3.28 (m, 4H), 2.43-2.49 (q, 1H), 2.1 (s, 1.5H), 0.9 (t, 1.5H).

### Example-11: Preparation of Co-Crystal of Empagliflozin and L-Proline from Compound of Formula (II-SH-A)

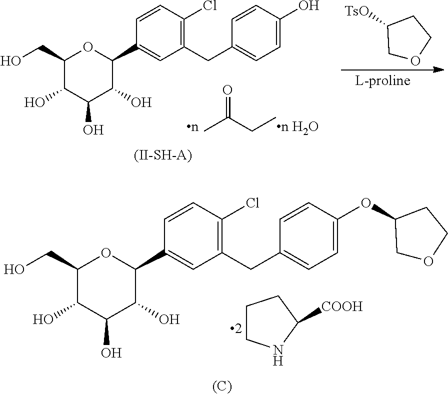

where n is 0.5

In a round bottom flask, 15 g compound of Formula (II-SH-A), 17.96 g cesium carbonate, 11.45 g (R)-tetrahydrofuran-3-yl-4-methylbenzenesulfonate and 150 mL DMSO or DMF were added at 25° C. to 35° C. The reaction mixture was heated to 35 to 40° C. and stirred for 4-5 hours. 2.86 g (R)-tetrahydrofuran-3-yl-4-methylbenzenesulfonate and 6.41 g cesium carbonate were added and the reaction to mixture was stirred at 40-50° C. for 24-36 hours. The reaction mixture then cooled to 25° C. to 35° C. 300 mL water was taken in another flask and cooled to 0-10° C. The reaction mixture was added into the water at 0-10° C. The reaction mixture was stirred at 25° C.-35° C. 75 mL toluene was added into the reaction mixture stirred for 30 minutes. The layers were separated and the aqueous layer was washed 2 to 3 times with toluene. In another flask, the aqueous layer and 450 mL methylene dichloride were taken and stirred for 30 minutes. Layers were separated. Organic layer was treated with 75 mL of 5% NaOH solution for 30 minutes and the layers were separated. The organic layer then treated with brine solution and then dried over sodium sulphate. The solvent was distilled out under vacuum to get a residue. 150 mL ethanol, 8 gm L-proline and 0.75 mL water were added into the flask having the residue and the reaction mixture was stirred for 30 minutes at 75-80° C. The reaction mixture then cooled and 75 mL toluene was added and stirred for 2 hours at 25-30° C. The solid was filtered and washed with ethanol and then dried to obtain co-crystal of empagliflozin and L-proline.

### Example-12: Preparation of Co-Crystal of Empagliflozin and L-Proline

In a round bottom flask, 10.0 g empagliflozin was dissolved in 300 mL ethanol at 25±3° C. To this solution, 5.6 g L-Proline and 0.5 mL water were added and the reaction mixture was stirred for 30 to 40 minutes at 75-80° C. to make a clear solution. The reaction mixture was stirred for 2 hours at 25-30° C. to get the precipitate. The precipitated mass was filtered and washed with 10 mL ethanol and then dried at 50 to 60° C. for 8 to 12 hours in an oven to obtain 8 g of co-crystal of empagliflozin and L-proline.

### Example-13: Preparation of Co-Crystal of Empagliflozin and L-Proline

In a round bottom flask, 10.0 g empagliflozin was dissolved in 200 mL ethanol at 25±3° C. To this solution, 5.6 g L-Proline and 0.5 mL water were added and the reaction mixture was stirred for 30 to 40 minutes at 75-80° C. to make a clear solution. The reaction mixture was cooled to 50-55° C. and 100 mL toluene was added. The reaction mixture was stirred for 2 hours at 25-30° C. to get the precipitate. The precipitated mass was filtered and washed with 10 mL ethanol and then dried at 50 to 60° C. for 8 to 12 hours in an oven to obtain 8 g of co-crystal of empagliflozin and L-proline.

### Example-14: Preparation of Co-Crystal of Empagliflozin and L-Proline

In a round bottom flask, 2.0 g empagliflozin was dissolved in 4.0 mL dimethylformamide at 25±3° C. To this solution, 1.12 g L-Proline and 1.0 mL water were added and the reaction mixture was stirred for 30 to 40 minutes at 30° C. to make a clear solution. The reaction mixture was stirred for 2 hours at 0-10° C. and the solution was seeded with co-crystal and stirred for an hour at 10° C. The reaction mixture was then stirred for an hour at 20-25° C. The precipitated mass was filtered and washed with pre-cooled 2 mL dimethylformamide and then dried at 50 to 55° C. for 8 to 12 hours in an oven to obtain 1.2 g of co-crystal of empagliflozin and L-proline.

### Example-15: Preparation of Amorphous Empagliflozin

In a round bottom flask, 10 g co-crystal of empagliflozin and L-proline, 100 ml ethyl acetate, 50 ml water and 20 ml tetrahydrofuran were taken at room temperature. The reaction mixture was stirred for 30 minutes at 25 to 35° C. The organic and aqueous layers were separated. The solvent was removed from the organic layer under vacuum at 45 to 55° C. The residue thus obtained was dissolved in 200 ml methanol at 25-35° C. The solution was spray dried to obtain amorphous empagliflozin.

### Example-16: Preparation of Amorphous Empagliflozin

In a round bottom flask, 10 g co-crystal of empagliflozin and L-proline, 100 ml ethyl acetate, 50 ml water and 20 ml methanol were taken at room temperature. The reaction mixture was stirred for 30 minutes at 25 to 35° C. The organic and aqueous to layers were separated. The solvent was removed from the organic layer under vacuum at 45 to 55° C. The residue thus obtained was dissolved in 200 ml methanol at 25-35° C. The solution was spray dried to obtain amorphous empagliflozin.

While the present invention has been described in terms of its specific embodiments, certain modifications and equivalents will be apparent to those skilled in the art and are intended to be included within the scope of the present invention.

1. A compound of general Formula (II-SH), 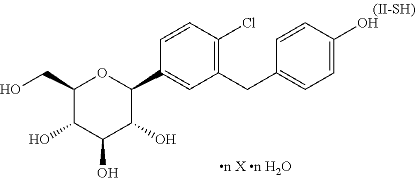
wherein n is 0.5 to 2, X is C₃ to C₆ ketone.

2. The process according to claim 18, wherein the compound of Formula (II-SH), is compound of Formula (II-SH-A), 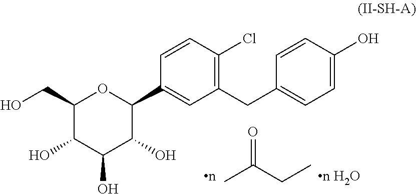
wherein n is 0.5 to 2.

3. The compound according to claim 2, wherein the compound is crystalline hemi methyl ethyl ketone solvate hemihydrate.

4. The compound according to claim 3, characterized by a powder X-ray diffraction pattern comprising peaks expressed in degrees 2θ at about 4.6, 9.8, 16.3, 16.7, 21.3, 22.1, and 24.1° 2θ±0.2° and ¹H NMR spectrum substantially as same as that depicted in FIG. 8.

5. A co-crystal of empagliflozin and amino acid selected from L-proline, L-threonine, L-cysteine, L-methionine, L-phenylalanine, L-tyrosine, L-asparagine, L-aspartic acid, L-glutamine, L-glutamic acid, L-lysine, L-arginine, L-histidine, L-serine, L-tryptophan, L-alanine, L-valine, L-leusine, L-isoleusine, D-aspargine, D-aspartic acid, D-glutamine, D-phenylalanine, D-alanine, D-valine, D-leusine, D-glutamic acid, D-arginine, D-serine, D-threonine, D-methionine, D-isoleusine and D-proline.

6. The co-crystal according to claim 5, is 1:2 co-crystal of empagliflozin and L-proline.

7. The co-crystal according to claim 6, is characterized by a powder X-ray diffraction pattern comprising peaks expressed in degrees 2-theta at about 4.3°, 12.9°, 15.6°, 18.7°, 20.0° and 21.6° 2θ±0.2° 2θ.

8. The co-crystal according to claim 6 is characterized by X-ray powder diffraction pattern substantially same as depicted in FIG. 1 and ¹H NMR spectrum substantially same as depicted in FIG. 2.

9. A process for the preparation of co-crystal according to claim 6, the process comprising:
(a) dissolving empagliflozin and L-proline in one or more solvents to obtain a reaction mixture;
(b) optionally warming the reaction mixture to obtain complete dissolution;
(c) cooling the reaction mixture; and
(d) removing the solvent to obtain the co-crystal.

10. The process according to claim 9, wherein the solvents comprises one or more of water, dimethyl formamide, dimethyl sulfoxide, dimethylacetamide, N-methyl pyrrolidone, ethanol, toluene, or mixtures thereof.

11. A process for the preparation of an amorphous form of empagliflozin, the process comprising:
(a) dissolving co-crystal of empagliflozin and L-proline according to claim 6 in one or more solvents;
(b) removing the solvents to obtain a residue;
(c) dissolving the residue in one or more another solvent; and
(d) removing the solvent to obtain the amorphous form of empagliflozin.

12. The process according to claim 11, wherein the solvents comprises one or more of water, C₁-C₄-alcohols, tetrahydrofuran, toluene, methylene dichloride, ethylene dichloride, carbon tetrachloride, ethyl acetate, dimethyl formamide, dimethyl sulfoxide, or mixture thereof.

13. The process according to claim 11, wherein removing the solvent comprises one or more of rotational distillation spray drying, agitated thin film drying (“ATFD”), or freeze drying (lyophilization).

14. A compound of Formula (VI) 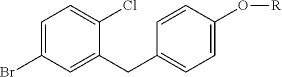
wherein R is a protecting group selected from trityl, allyl or tetrahydropyran.

15. A compound of Formula (VI-a)

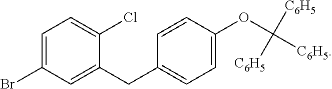

16. The compound according to claim 15, is crystalline characterized by a powder X-ray diffraction pattern comprising peaks expressed in degrees 2θ at about 5.3, 10.5, 19.8, 21.0, 22.0, 25.0 and 26.3° 2θ±0.2° 2θ.

17. A compound of Formula (IV-P)

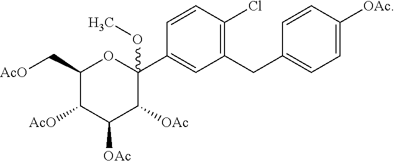 [SMILES: COC1(c2ccc(Cl)c(Cc3ccc(OC(C)=O)cc3)c2)O[C@H](COC(C)=O)[C@@H](OC(C)=O)[C@H](OC(C)=O)[C@H]1OC(C)=O]

18. A process for preparing the co-crystal of empagliflozin and L-proline according to claim 6, comprising reacting a compound of the Formula (II-SH) with L-proline and (R)-tetrahydrofuran-3-yl-4-methylbenzenesulfonate, 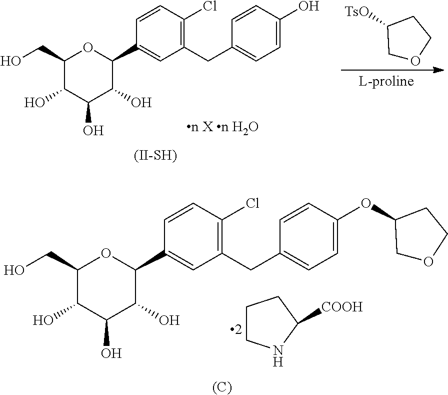
wherein n is 0.5 to 2, X is C₃ to C₆ ketone.

19. A process for the preparation of empagliflozin, the process comprising:
(a) reacting 5-bromo-2-chloro-4′-ethoxydiphenylmethane of Formula (VIII) with Lewis acid to obtain 5-bromo-2-chloro-4′-hydroxydiphenylmethane of Formula (VII); 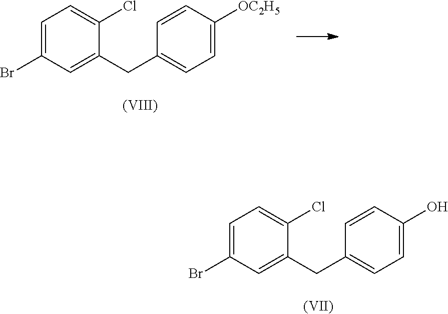
(b) reacting 5-bromo-2-chloro-4′-hydroxydiphenylmethane of Formula (VII) with a hydroxy protecting reagent to obtain a compound of Formula (VI); 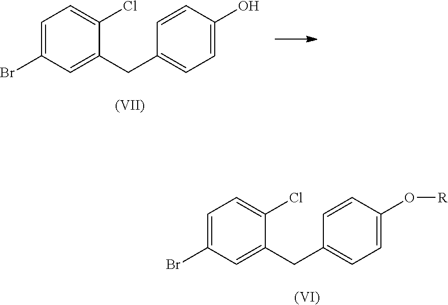
(c) reacting the compound of Formula (VI) with a compound of Formula (P-G) to obtain a compound of Formula (V); 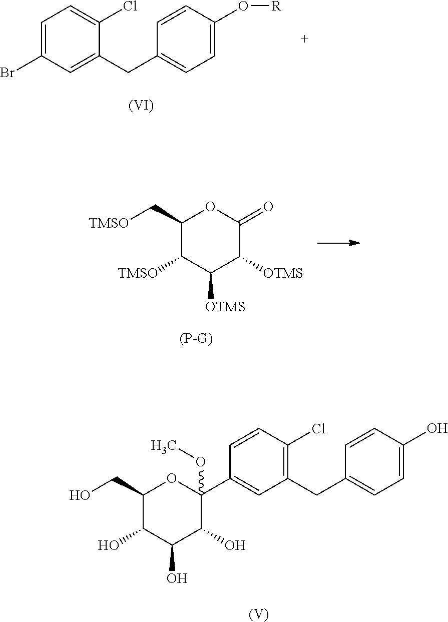
(d) converting the compound of Formula (V) into a compound of Formula (II-SH); 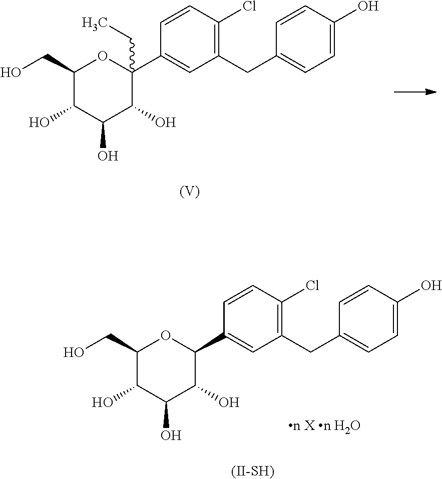
wherein n is 0.5 to 2, X is C₃ to C₆ ketone,
(e) converting the compound of the Formula (II-SH) into 1:2 co-crystal of empagliflozin and L-proline; and 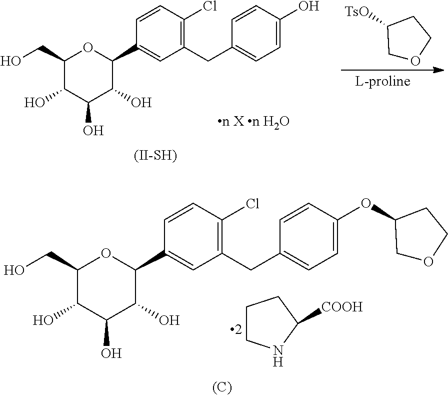
(f) converting the obtained 1:2 co-crystal of L-proline into empagliflozin.

20. Empagliflozin substantially free from one or more of impurities selected from diastereomer impurity, dialkylated impurity, furoanose impurity-1, Furanose impurity-2 and monoacetylated empagliflozin as determined by area percentage of HPLC.

21. A pharmaceutical composition comprising amorphous empagliflozin substantially free from one or more impurities according to claim 22 and one pharmaceutically acceptable excipients, diluents or carriers.

22. A pharmaceutical composition according to claim 21 for the treatment of diabetes.
